(19) United States
(12) Patent Application Publication
(10) Pub. No.: US 2021/0106282 A1
(43) Pub. Date: Apr. 15, 2021

(54) ERGONOMIC HELD WEIGHT UNITS, RELATED COMPUTING DEVICE APPLICATIONS AND METHOD OF USE

(71) Applicant: Christian Malcolm, Thousand Oaks, CA (US)

(72) Inventor: Christian Malcolm, Thousand Oaks, CA (US)

(21) Appl. No.: 17/033,606

(22) Filed: Sep. 25, 2020

Related U.S. Application Data
(63) Continuation of application No. 16/241,951, filed on Jan. 7, 2019, now abandoned.
Application No. 16/241,951 is a continuation of application No. 15/244,908, filed on Aug. 23, 2016, now abandoned.
Application No. 15/244,908 is a continuation-in-part of application No. 15/188,048, filed on Jun. 21, 2016, now Pat. No. 10,223,557.

(51) Int. Cl. A61B 5/00; A63B 69/00; A63B 71/06; A61B 5/11; A63B 21/02; A63B 21/065; A63B 21/00; G06F 1/16; G06F 3/01; H04M 1/72403

(52) U.S. Cl. CPC A61B 5/6895; A63B 71/0036; A63B 71/0622; A61B 5/1118; A63B 21/028; A63B 21/065; A63B 21/4039; G06F 1/1694; G06F 1/163; G06F 3/011; H04M 1/72403; G06F 1/1626; A63B 2230/75; A61B 5/0022; A63B 69/0028

(57) ABSTRACT

Hand held weight units of light weight manufactured as a solid unit, a shell unit with core insert combinations or modular units with interlocking ends. Shell units with core inserts and modular interlocking units allow for the changing of held weight by inserting or removing inserts or by locking or unlocking of modular weight unit sets creating varying held weight. The weight units are primarily used with upper body exercises during aerobic exercises in the home, outdoors, or in a gym setting such as walking or running to vary the intensity of workout during use.

### CROSS-REFERENCE TO RELATED APPLICATIONS

This application is a continuation of U.S. patent application Ser. No. 16/241,951, filed Jan. 7, 2019, which is a continuation of U.S. patent application Ser. No. 15/244,908, filed Aug. 23, 2016, which is continuation-in-part application of U.S. patent application Ser. No. 15/188,048, filed Jun. 21, 2016, now U.S. Pat. No. 10,223,557, the disclosures of each of which are hereby incorporated by reference in their entireties for all purposes.

### BACKGROUND OF THE INVENTION

The present invention relates to dumbbells, adjustable dumbbells, and more particularly to lightweight dumbbells or lightweight adjustable hand weights having specific ergonomic shapes that allow for the centering of weight in the palm of the hand, and interlocking features that allow for the ease of combination of units during an aerobic exercise to achieve a specific desired weight and intensity of workout.

Moreover, the present invention also relates to worn and other computing devices that are intended to track heart rates, steps or strides, body movements, forces and exertion of movements with handheld weights and calories burned per time unit through the use of: heart rate monitors, pedometers, gyroscopes, accelerators and other sensors.

Aerobic exercise has always been an effective way to lose weight, increase physical conditioning and maintain a healthy lifestyle. However, over time, the body adjusts to the aerobic exercise by increasing both strength and physical endurance, making the same activity easier and easier to perform. Therefore, to maintain a high level of physical exertion during aerobic exercises, weight or resistance must be added or increased over time to keep up with the body's increased physical conditioning.

Adding handheld and other weights to any aerobic activity is a great way to accomplish such addition of weight or resistance. There exists a wide variety of indoor and outdoor exercises that are made more challenging when even the smallest amount of held weight is added to the activity. Increasing the heart rate, muscle activity and total calories burned per hour is possible with the addition of dumbbells or various methods of holding weight in the hand during physical activities.

A conventional dumbbell is intrinsically formed as a single solid unit and its weight is constant. Hence, devoted users typically possess a multitude of dumbbell sizes and shapes of different weights and, in doing so, must choose a single weight to hold during prolonged aerobic exercise. This single weight limitation poses a problem for the users doing interval upper body weight training during continuous or prolonged aerobic exercises, such as walking or running, as it forces the individual to use one weight across all upper body exercises.

A typical adjustable dumbbell system is intrinsically designed with a method of adding plates, rings or other weight segments to a center bar or grip. Hence, users need to add or subtract weight segments and then lock or screw in the weight in place prior to aerobic exercise. Changing the weight during aerobic exercise would require unlocking of weight segments from the central bar or grip and adding or subtracting weight, then relocking the new desired weight into place. In doing so, the wearer must stop physical activity and make the changes to each dumbbell before resuming the activity. In addition, the typical adjustable dumbbell weight isn't practical to carry on the body during prolonged aerobic exercise and lends itself for use only within the home or gym setting where the additional weight segments are properly stored or housed on racks or in a complete set of incremental weight stacks. Hence, users must typically choose a single weight to hold during prolonged aerobic exercise which poses a problem for the users doing interval upper body weight training during continuous aerobic exercise.

Simply put, using the same held weight across many different upper body exercises or arm movements does not achieve the most beneficial workout when compared to alternating intensities of exercises by changing the held weight during aerobic exercises. In addition, as previously mentioned, traditional dumbbells and adjustable dumbbells are formed with a center grip bar with weight blocks attached at both ends, the larger the weight the larger the outer weight blocks or segments become. Therefore, these traditional dumbbell shapes are not advantageous during aerobic activity, as they often alter the form of the exercise to limit the risk of banging the weights together or coming in contact with the body. In addition, traditional dumbbell shapes can come in contact with aerobic machines such as treadmills or step climbers during certain arm movements or motions, can get hung up or caught on headphone wires worn by the user during exercise and can hit a walking or running partner's dumbbell or body as they are swung back and forth by the holder when exercising in a group. Therefore, there exists a need for a hand held weight system in which most of the weight is centered in the palm of the hand. In addition to the single weight unit being centered in the palm, it is also advantageous for the user that when units are joined together and held as one joined unit of increased weight and mass, there is only a limited amount of weight unit exposed on either end of the holder's hand grip circumference.

There may be additional benefits having the weight centered over or otherwise held in the palm in a rounded or oval shape. Discover Walking and The Walking Site both point out that walking with hand weights or dumbbells can increase blood pressure caused by the gripping or squeezing of the weights tightly. “If an individual carries weights several days per week for an extended period of time, their blood pressure could be adversely affected. High blood pressure can lead to other serious health problems such as heart disease and an increased risk for stroke,” The Walking Site. A rounded egg or oval shaped weight unit, with one or more finger grips centered over the palm, can help reduce the tendency for the user to clench their first tightly around a thin centered bar grip and thus can reduce or eliminate the risk of increasing blood pressure during aerobic activity. Therefore, when an individual chooses to walk or run with weights for the added training benefit, it is most advantageous to hold a weight that is specifically designed to be ergonomically centered in the palm of the hand and for a light gentle holding thereof during prolonged aerobic exercise. In addition to all the physical intrinsic benefits of holding a light weight that is centered in the palm of the hand, for some individuals, the ability to partially or fully conceal the light weight in the center of the hand is advantageous. Some individuals perceive the holding of standard dumbbells more visible and unpleasant, causing them to feel self-conscious about exercising with held weights. Additionally, women or men with long finger nails can experience a digging in effect when gripping a bar of traditional dumbbells, which in some instances has a small circumference.

Individuals who use adjustable held weight during prolonged aerobic exercise to increase or decrease the intensity of their workout, can benefit greatly by tracking, monitoring and reviewing the intensity of their work out across one, multiple or all muscle groups used during exercise. To facilitate these benefits, data can be generated by tracking information monitored by sensors coupled with or integrated into the held weight with regard to specific exercises. As such, each muscle group associated with the exercise can be critically reviewed by users, coaches or healthcare professionals in order to understand, monitor and plan to improve their individual progress and performance over time. Therefore, it can be critical to provide this data in an easy to understand and reviewable format. In order for the user to set and achieve his or her goals of physical fitness, they should be able to comprehend which exercises they have performed and how the exercises correspond to different muscle groups, as well as how the exercises are impacted by the held weight.

Small to medium sized dumbbells are often used in physical or occupational therapy to help strengthen muscle groups, increase blood flow, stretch, and regain motion. In addition to the above stated advantages of a palm centered weight unit for use during aerobic exercises and the need to track important data in a useful format, there also exists the need for a small to medium size therapeutic weight unit with a standard centered tracking sensor package merged together for therapeutic purposes.

A weight unit with a centralized insert or otherwise physically coupled tracking sensor package may be more advantageous to use in place of a traditional dumbbell, since users can track real time motions of prescribed therapeutic exercises. Weight unit sizes may generally be palm sized and centered to provide for an added benefit, namely that the weight does not exceed the circumference of the hand. Alternatively or additionally, weight units may be specific custom shapes unique to physical or occupational therapy.

Hand held adjustable light weight dumbbells, gloves with adjustable weight bars that encircle a hand and other similar light weight hand held systems are designed to increase or decrease held weight prior to an aerobic exercise and are generally known in the art. U.S. Pat. No. 4,351,526 to Schwartz, U.S. Pat. No. 5,250,014 to Chang, U.S. Pat. No. 6,042,508 to Clem, U.S. Pat. No. 7,025,713 to Dalebout, U.S. Pat. No. 7,908,672 to Butler, U.S. Pat. No. 8,684,893 to Tang, U.S. Pat. No. 8,992,396 to Wang, U.S. Pat. No. 9,132,316 to Lima, variously disclose different types of such adjustable dumbbells, gloves and weight systems which in some instances, include tracking or monitoring devices within. However, none of these devices is satisfactory for extended aerobic exercises in which hundreds or even thousands of repetitive arm movements are performed, where each movement may have its own unique weight tolerance or desired weight due to differences in required movement, muscle group and fitness level of the user. To elaborate, an individual of average physical conditioning can perform bicep curls while engaged in an aerobic activity such as walking or jogging at a specific weight. However, if the individual wishes to change an upper body arm movement to achieve a more well-rounded upper body work out, the weight may need to be increased or decreased. Examples of these movements include arm circles, fast punching jabs, shoulder presses, uppercuts and others. Using the same weight across these varying upper body exercises and muscle groups is not advantageous and can be disadvantageous for achieving optimized workouts. A more effective course for building and/or toning muscle groups during prolonged aerobic exercise is to alter the intensity of the exercise by changing the held weight across repetitions and sets of repetitions for varying exercises and muscle groups.

Therefore, there exists the need for a hand held weight system where weight units, weight inserts or other weight related apparatuses can be stored on the body and easily locked or linked together during aerobic exercise in order to increase or decrease the intensity of held weight during upper body and full body exercises.

In addition to the adjustable weight system, the embodiments described herein also relate to worn tracking or monitoring devices that can include use of one or more pedometers, accelerators, gyroscopes and other sensors, alone or in combination, to track a user's motion and physically exerted output during exercise. These devices have previously been known in inferior prior art disclosures, as shown in U.S. Pat. No. 7,379,770 to Szeto, U.S. Pat. No. 8,579,827 to Rulkov, and U.S. Pat. No. 9,237,855 to Hong, which variously disclose different types of monitoring and tracking devices. Such devices have several common primary functions, including: 1.) tracking and displaying the heart rate of a user or wearer using sensors; 2.) tracking and displaying estimated or actual calories burned during activity and/or while at rest using the wearer's heart rate compared to variables inputted by the user, such as: weight, fitness level and age; and 3.) displaying overall performance of an activity or exercise over time and setting future goals or targets for physical activity. One example of this functionality is measuring how far an individual ran during an hour, the individual's average heart rate during the time, the individual's high and low heart rate achieved during the activity and how many calories the individual burned during that activity.

Although the above mentioned devices allow users to track and display metrics of physical activity and exertion of the wearer including: heart rate, calories burned, number of steps, strides, or cycles, none are optimized for tracking and monitoring various physical activities performed in combination with variable held weights and variable upper body exercises during prolonged aerobic activities.

Therefore, there exists the need for: user interfaces including manual and audiovisual features of a tracking device (such as a smart watch, smart phone or other device) that allow a wearer to input and change a variable held weight into the tracking device during prolonged aerobic exercise to accurately track held weight with upper body exercises and an automatic tracking sensor system that is embedded or coupled with the tracking device that can read, monitor and track the variable combinations of held RFID (Radio Frequency Identification) or other tagged or chipped weights used during upper or full body exercises during aerobic activity, examples of which include: walking, jogging, swimming, running, yoga, stationary exercise, stretching, martial arts and others.

### SUMMARY OF THE INVENTION

Provided herein are embodiments of systems, devices and methods for manufacturing and using small, lightweight interlocking modular dumbbell and modular weight units to allow a user to change or modify the hand held weight easily by locking, unlocking or otherwise adjusting weight units during aerobic or other exercise, and in some embodiments include user device integration for tracking sensors associated therewith.

In some therapy related embodiments, an injured patient can be required to use weight and strength training in order to heal. As the patient performs various exercises and the injured body part heals, the patient is able to remove a sensor unit, held within the body of an exercise monitoring unit, and place it inside or otherwise couple it with a next higher weight unit before continuing their prescribed physical therapy. This can be an iterative process that is repeated until the patient is healed. Doctors, physical therapists and other health care providers may purchase and lend such a system or “kit” to patients, or patients can purchase, lease or rent the kit on their own. Varying therapeutic weight units, interchangeable monitoring sensor units and network connected computing devices, combined with tracking applications and web portals can encourage patients to do physical therapy “homework” and prescribed exercises to improve recovery times and reduce visits to physical and occupational therapy offices.

The embodiments disclosed herein mitigate and eliminate problems with the prior art by providing significant improvements. The methods, systems and devices for locking and unlocking weight segments may use multiple means of joining or coupling objects together including male and female locking pins, interlocking rigid features, magnets and others. Another object illustrated and described with regard to the various example embodiments is to provide a small, lightweight unit made from lead, cast iron, steel or other heavy or durable metals or materials to provide the desired weights and shapes of the units. Additionally, an inner core and an outer core of varying metals or casted layers can be provided within the modular unit to achieve the specific weight and shape of the units, as well as to create a barrier or protective layer around metals or materials that may be denser but softer, such as lead. Additionally, provided herein are embodiments including one or more RFID or other tracking tags, microchips or other tracking devices or systems that can be embedded within or otherwise coupled to a weight unit and that can be communicatively coupled to a user device, such as a smart phone, smart watch or others. These can automatically identify the user, individual weight values, combined continuous weight values and other data or information during upper body movements and exercise. In some embodiments, one or more modular weight units are provided that can be centered in the palm of a user's hand alone or in combination as a set, or as a shell weight unit with an optionally removable insert.

It is another object of the present invention to provide a less expensive non-interlocking weight unit or hand dumbbell that can be centered over the palm of a user's hand, which can be used during aerobic or other exercises with and without tracking weight tags, since some individuals may benefit greatly from a palm centered weight but may not require the added benefit of combined weights. For example, older individuals, individuals with disabilities, individuals new to physical activity or individuals with injuries may only be capable of utilizing a singular, solid, light weight unit and may not need an interlocking high intensity workout.

Some embodiments allow a user to utilize a palm-centered weight shell with varying and optionally removable insert weights that may be locked therein or removed to increase or decrease weight accordingly during aerobic exercise. It is another object of the present invention to utilize motion tracking sensors, which can include one or more gyroscopes, accelerators or others within a smart watch or other wearable monitoring device for a user's wrist or forearm, and which may, in combination with inputs from the wearer provide data points of each movement performed by the user. One example a user inputting their height, which can be used in combination with the other wearable device to track individual upper body motion of the wearer by executing algorithms to compute X, Y and Z axis points (roll, pitch, and yaw) and rotational acceleration. In some embodiments, devices can be provided in order to store the weight units or inserts on the body during aerobic exercise until needed. Such storage devices can include, but are not limited to: upper body vests, waist belts, arm bands, ankle bands and other storage systems related to the ergonomic storage of weight units during aerobic exercise.

In some embodiments, devices can utilize and execute software, stored in non-transitory computer readable memory or otherwise installed on or downloaded to one or more monitoring or tracking device, that enable users to enter one or more of: total body held weight including hand held weight and any stored weight by use of a vest, belt or other method at the beginning of exercise. Then, as the user increases or decreases hand held weight and increases or decreases stored weight on the body, the software can track the changes in movement and results through communication either by automated tagging systems, manual or voice commands or others, in order to accurately measure both hand held weight affecting upper body movements and weight held on the body that can impact upper, lower or full body muscle groups accordingly.

As described herein, systems methods and devices are provided that users can utilize, including weight units as therapeutic objects during physical or occupational therapy. Small units of held weight that can be centered in the hand can prove to be advantageous when stimulating torn or damaged muscles, tendons, or ligaments than traditional dumbbells that require a greater grip or squeezing effect to hold and perform therapeutic movements. As such, it is another object of these embodiments to provide use of the light weight palm centered units in high frequency movements in order to increase the intensity of exercise during both stationary activity or aerobic exercise, such as boxing, martial arts or other fast or repetitive hand movements. It is another object of the present invention to use the light weight palm centered units in low frequency, slow movements to increase the intensity or effectiveness of the activity, such as stretching, yoga, meditation, and tai chi, in order to benefit from the addition of palm centered weights.

Some embodiments can include one or more of: light weight hand straps, Velcro or hook and loop wraps, neoprene grips, rubber flexible grips, rubber gel filled grips, gloves or other apparatuses, in order to secure and hold the weight unit centered in the palm during high speed arm movements. Arm movements generated from other activities, such as sprinting or swimming, where a user's hand is not generally closed around the weight unit can also benefit in some embodiments.

Various embodiments include weight units coated, covered and/or coupled with one or a combination of: a resilient plastic, neoprene, or rubber material to ensure the proper gripping or holding thereof during aerobic exercise. These can also decrease a slipping effect caused by an accumulation of sweat build up during activity, allow for the easy cleaning of the weight units with soap and water after use, protect weight unit chips or tags from damage during use or cleaning, and protect the weight units from being scratched, scraped or otherwise damaged if dropped, hit or otherwise impacted other objects or surfaces. Sweat resistant covers or wraps can be crafted from resilient plastic or rubber in various colors or prints in specific locations, to provide suggested gripping points for users as well as marketing or branding opportunities. Examples of branding or marking opportunities include: company logos or slogans, university colors and logos, colors symbolizing special events, team logos and other prints or colors that may be special or meaningful for users.

Data collected from sensors such as gyroscopes and accelerator units can be used in conjunction with the continuous held weight values to calculate various metrics that can be displayed for users on smart monitoring devices, uploaded or transmitted to a smart phone, tablet, laptop or other computing device in a manner that is easy to read and understand. Formatting can include: graphs, charts, totals of arm or other movements by category and muscle group, metrics of pounds lifted per hour, pounds lifted per muscle group, total pounds lifted, total pounds lifted per individual exercise and numerous others. This data and calculations based on the data, in conjunction with the varying held weight, can provide be used to provide an overall analysis displayed on smart monitoring devices or uploaded to laptops, tablets, phones or other computing devices to depict an animated male or female digital body display that can include the intensity of the exercises and the muscle groups used to perform said exercises identified by color of intensity and performance. For example, if an individual performed mostly all bicep curls during their aerobic activity, the digital body can show red in the bicep muscle for high intensity, yellow in the forearm muscle group for medium intensity and green for low performance or intensity in the remaining upper body muscle groups. Furthermore, by rotating the digital body with a swipe of a finger on a user interface, such as a display window, the digital body can rotate to show muscle groups located on the individual's back and provide a complete the entire upper body muscle groups. The digital body may also include lower body muscle groups, utilizing data generated from the sensors to track muscle activity during aerobic exercise such as walking, jogging, running, climbing stairs, hiking or others.

Various devices and components described herein can include a power saving mode, in order preserve battery power by monitoring the held weight only when in use. As such, they can be optionally powered on and powered down into a sleep mode by automatically or manually awakening only when a motion or continuous movement is altered or changed in such a way that it signifies a possible addition or subtraction of held weight or exercise starting/stopping. For example, an individual can begin to walk with one pound of held weight and is performing bicep curls to warm up their upper body, so the sensor initially tracks the one pound and then the device may go into sleep mode. After a few minutes the individual may stop arm movements to add another weight segment or insert increasing the value of a held weight to two pounds, at which point the tracking system is awakened due to the change monitored by the sensors based on an identified break or change in user movements. This can trigger the device to check for a weight adjustment and identifies an increased value in held weight for all future arm movements until another break in movement occurs and so on.

Digital body displays can be used to teach and help users develop an exercise routine by a simple user friendly mode in the monitoring device, smart phone, tablet, laptop or other computer that is linked to a user's profile and history in a web or application portal. For example, if a user wishes to work on triceps training, the user can open a training mode on a user interface of the device and point, tap, command or otherwise interact with and select the specific muscle or muscle group on the digital body display, whereafter the digital body can provide several arm movements to complete during aerobic exercise that target the specific region identified. Small locking light units, each powered by one or more coupled batteries, may be specifically designed to attach or otherwise couple to the ends of each weight unit by use of a screw system, magnet, friction locking, or other system to illuminate a user's road or pathway, signal to traffic or otherwise light and indicate that an individual is present on the road or pathway during dimly lit or dark conditions.

The ability to train users in a correct or desired therapeutic movement, through use of digital display units such as smart phones, tablets or other display may be very advantageous during physical or occupational therapy, especially when coupled with real time body tracking through the use of sensors placed within therapeutic weights. Therapeutic weight units that have a recessed center area can be fitted with a rechargeable and optionally removable wireless sensor package equipped with various real time tracking and monitoring devices, including but not limited to: gyroscopes, multiple directional accelerators or others, to aid and track therapeutic motions with desired weight.

Data gathered by the inserted sensors can be transferred to a smart phone, tablet, or another display unit wirelessly to depict real time hand, arm, or body motions with the held weight for therapeutic purposes. Additionally, by the use of straps or belts, the weight units or individual weight segments may be fixed to the body to ensure the proper location of the held weight during therapeutic movements. Furthermore, a specialized, ruled mat or paper aids the therapist to quickly identify the width, length, and circumference of the user's body part by simply placing the body part onto the paper and taking several pictures from different angles.

A physical therapist, trainer or coach may also take measurements of the body part and input it directly into the user's profile. Once the body part's dimensions are identified, the therapist can identify any number of injuries to the area and create a treatment plan. The treatment plan can use a number of stored algorithms to create the digital body part's therapeutic movement in the exact form that is prescribed by the therapist based on the data provided.

Additionally, daily, weekly or monthly summaries of therapy activity may be uploaded in real time via a computer network to the therapist for analysis and confirmation of prescribed patient movement. As people continue to live active lives as they age, an increased risk of damage to their body under stress and exertion exists due to physical activity. The US Bureau of Labor and Statistics predicts, “A job outlook increase in both physical and occupational therapy jobs from 2014-2024 of 34%, much higher than the national average, with an average yearly salary of $84,020 in May of 2015, almost twice the national wage average.” As such, the demand for physical and occupational therapists on the rise in the next decade along with the already high cost of individual therapy, there exists the need for a device for patients to get reliable and accurate therapy without the need to visit a therapist so frequently.

The configuration of the devices described herein in detail are only example embodiments and should not be considered limiting. Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. Therefore, it should be appreciated that these drawings depict only embodiments of the invention and are not to be considered limiting of its scope through the use of the accompanying drawings, in which:

FIG. 1 is an isometric view of a modular weight unit in accordance with the present invention;

FIG. 2 is an additional isometric view of a modular weight unit from a rotated perspective;

FIG. 3 is a perspective view of two modular weight units joined or locked together in a combined set;

FIG. 4 is an additional perspective view of two modular weight units joined or locked together from a rotated perspective;

FIG. 5 is a cross section of the male and female interlocking embodiment method of the present invention;

FIG. 6 is an alternative cross section of the male and female interlocking embodiment method of the present invention;

FIG. 7 is an alternative interlocking method featuring interlocking rigid structures of the present invention;

FIG. 8 is both a perspective view of an alternative interlocking weight unit and a close up of an alternative interlocking method featuring interlocking rigid structures of the present invention;

FIG. 9 is both a perspective view of an alternative weight unit shell and a cross sectional view of the alternative preferred embodiment of the present invention;

FIG. 10 is a perspective view of both the shell weight unit and matching insert of the alternative preferred embodiment of the present invention;

FIG. 11 is a perspective view of the shell weight unit with matching insert within, and a cross section view of the weight unit and insert as a combined set of the present invention;

FIG. 12 is a perspective view of an alternative singular non-interlocking weight unit with a flat end and no finger grips;

FIG. 13 is a perspective view of an additional singular non-interlocking weight unit with rounded ends and finger grips;

FIG. 14 is a perspective view of an alternative singular non-interlocking weight unit with rounded ends and without finger grips;

FIG. 15 is a perspective view of an additional alternative singular non-interlocking weight unit with a rounded body and without finger grips;

FIG. 16 is a perspective view of the modular weight unit held centered in the palm of the hand in a singular unit use, non-interlocking weight unit.

FIG. 17 is a perspective view of the modular weight unit held centered in the palm of the hand in a combined set;

FIG. 18 is a perspective view of the modular weight unit and the rubber grip or glove of the modular weight separately;

FIG. 19 is a perspective view of the modular weight unit and the rubber grip or glove of the modular weight combined;

FIG. 20 is a perspective view of the weight unit sleeve with longer extended finger grips;

FIG. 21 is a perspective view of the weight unit sleeve with a malleable soft grip sleeve;

FIG. 22 is a perspective view of a glove with a weight unit pocket to hold the weight unit in place during high frequency, fast upper body movements;

FIG. 23 is a perspective view of the modular weight unit stored on the body by use of a waist belt;

FIG. 24 is a perspective view of the modular weight unit stored on the body by use of a vest;

FIGS. 25, 26 and 27 respectively are perspective views of various modular weight units being tracked or monitored and coupled with a worn device by use of a manual, audio, and automated sensor tracking RFID tag respectively;

FIG. 28 shows several examples of established mathematical equations or algorithms used to determine the force, and calorie unit burned;

FIGS. 29A-29C are three flow diagrams showing the preferred methods of tracking and inputting held weight values of the present invention while tracking and monitoring upper body arm movements during aerobic exercise;

FIG. 30 is an example of the preferred embodiment of the present invention detailing a digital body display and data display example of the total b292ody summary either during or after aerobic exercise on a smart phone, tablet or similar device;

FIG. 31 is an isometric view of a modular therapy weight unit in accordance with the present invention, including the sensor insert combined as a set;

FIG. 32 is an alternative isometric view of a modular therapy weight unit in accordance with the present invention, including the sensor insert depicted separately;

FIG. 33 is an example of the preferred embodiment of the present invention, detailing the wireless communication between the sensor insert within the therapy weight unit to a smart phone, tablet or another display device;

FIG. 34 is an example of the preferred embodiment of the present invention, detailing a digital hand and upper body display of suggested movements, repetitions, and specific weight to be used during physical therapy on a smart phone, tablet or similar device, to be followed or mimicked by the intended user for therapeutic purposes;

FIG. 35 is an example of the preferred embodiment of the present invention detailing a digital body display and data display example of the total therapy summary of exercises or movements indicating performance, accuracy, and areas of necessary improvement for therapeutic purposes;

FIG. 36 is an example embodiment of a network system architecture diagram;

FIG. 37 is an example embodiment of a system server architecture diagram;

FIG. 38 is an example embodiment of a user device architecture diagram.

### DETAILED DESCRIPTION OF THE INVENTION

The foregoing and other objects, features, and advantages of the present invention will best be understood from the following description, the appended claims, and the accompanying drawings in which;

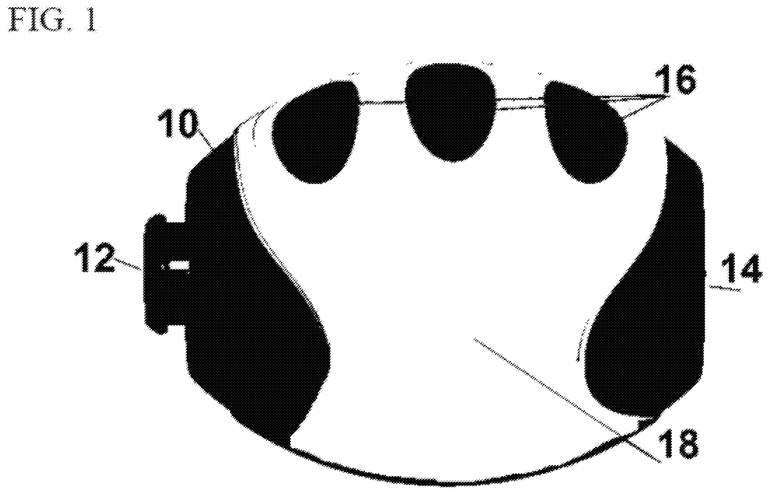

FIG. 1 is an isometric view illustrating a hand held modular weight unit 10. The modular weight unit includes several preferred embodiments including: the handheld weight unit's interlocking features 12, and 14, a set of ergonomic finger grips 16, and a rubber sleeve or griping cover 18. The two locking or latching devices, 12, and 14 are depicted here as a male and a female interlocking set capable of being twisted together into a locking or anchoring position to hold the two weight units together during use. Not depicted here is the potential use of one or more magnets to help lock the units together and hold them securely in place during use, as well as, allowing for the quick joining of the two weight units by magnetic force. The addition of the magnets may be a separate or secondary locking mechanism for security or may replace the male and female locking features entirely. The set of finger grips illustrated within as 16, and depicted as three individual finger indentations on the weight unit, 10, may be constructed with any number of finger grips from zero to 4 to align the weight unit in the center of the hand, and to promote the gentle holding thereof during use. The number, shape, and size of the finger grips will be determined by several contributing factors: the weight of the unit, the circumference and diameter of the weight unit, and the average size of the intended user's hand. Moreover, the inventor envisions that in order to satisfy the varying finger and hand sizes of users, it may be advantageous to have grip sizes, shapes, and counts that vary as well, depending on the fit and grip of the intended user. The rubber or silicone based sleeve or gripping glove, 18, is intended to assist users in achieving the exact fit and function desired as well as act as an anti-slip feature of the present invention, especially during prolonged exercise. Similarly, to the finger grip variety, there may be many differing sizes, shapes and materials such as rubber gels, soft, or hard silicone materials to create a multitude of fits and grips for the user. In addition, a silicone gel sleeve may wrap around the entire weight unit, not pictured, allowing the user to custom mold his/her hand to the grip itself making the fit adjustable and soft to the touch.

It should be understood that different weights can be used in various embodiments. In some embodiments these can include weights of different sizes, shapes and increments. As such, one weight can be 0.75 pounds, 1 pound, 5 pounds or others. Traditional dumbbell shapes can be used, egg weight shapes can be used that don't exceed the palm of the hand or other shapes in various embodiments and can be dependent on the intended exercises and physical therapy regimens.

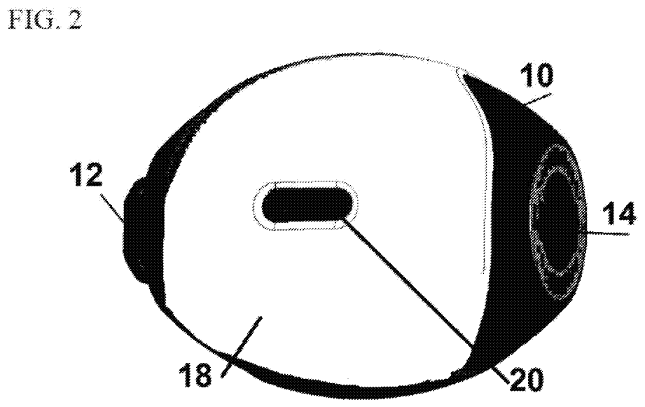

FIG. 2 is an alternative isometric view of the present invention within FIG. 1 and highlights both the male and female locking mechanism, 12 and 14, as well as, the potential use of a RFID tag, 20, and placement thereof. A radius is applied to the edge of the male and female locking mechanism to ensure that users do not pinch their skin when joining the male locking feature 12 together with the female mechanism 14.

The weight of the unit, and material used will determine the differing diameters and lengths of the weight unit. Heavier weight units may have larger overall diameters or lengths but all will have identical locking mechanisms to ensure that varying sizes of weight units may be combined together without difficulty during exercise to alter the intensity of the exercise by changing the held weight. Additionally, light weight units may have a smaller circumference and length or the same circumference and length with a hollowed out center to achieve the desired weight and mass.

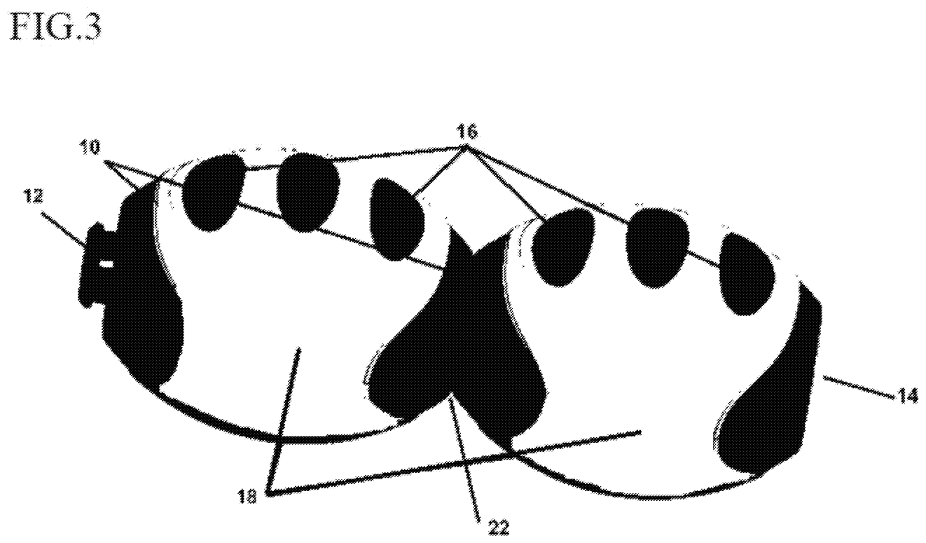

FIG. 3 is an isometric view of the present invention described in FIG. 1 combined into a set. As previously described, the weight units are joined together by means of locking male mechanism 12, and female mechanism 14, locked together as 22. The weight units are rotated to create the locking or anchoring feature which may or may not be supported by the used of one or multiple magnets holding the weight units together by means of magnetic force.

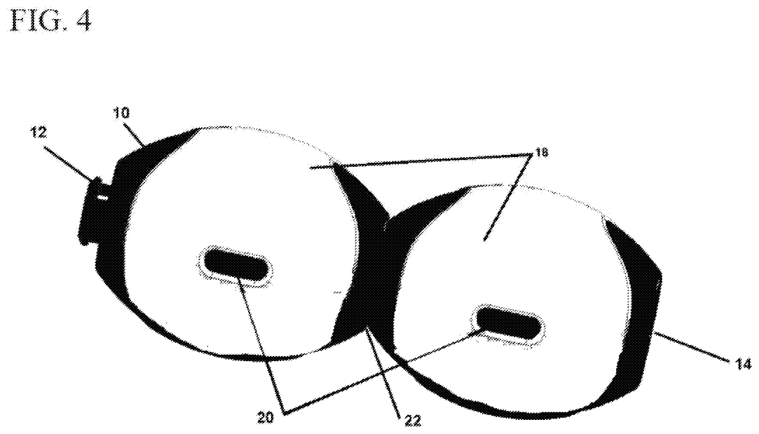

FIG. 4 is an alternative isometric view of the present invention depicting the back half of FIG. 3, highlighting each RFID tag, 20, on the identical weight units, 10. The RFID tags or chips are placed on the back side of the weight unit and opposite to the finger grips to allow for the ease of tracking of the weight units by a smart watch or other tracking device, 74.

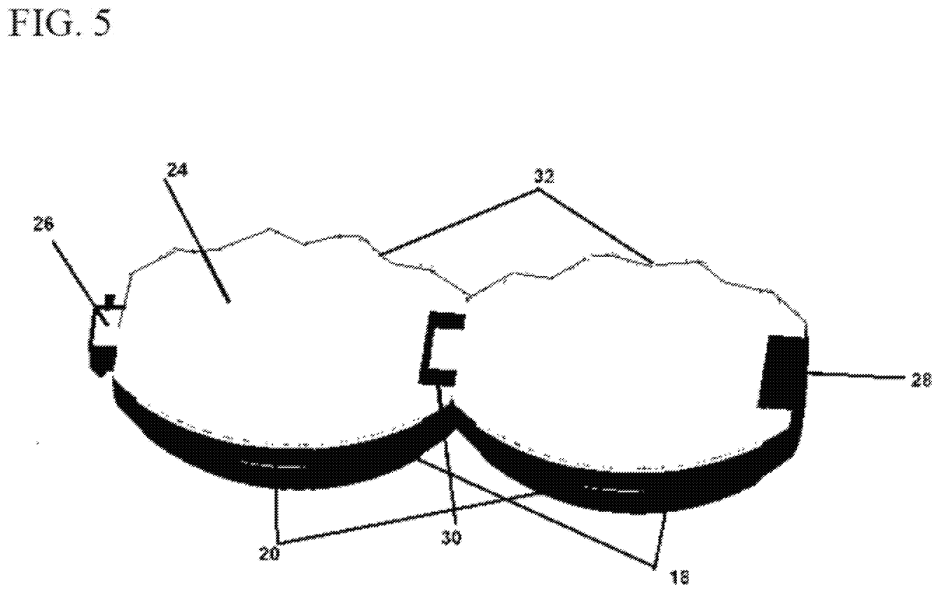

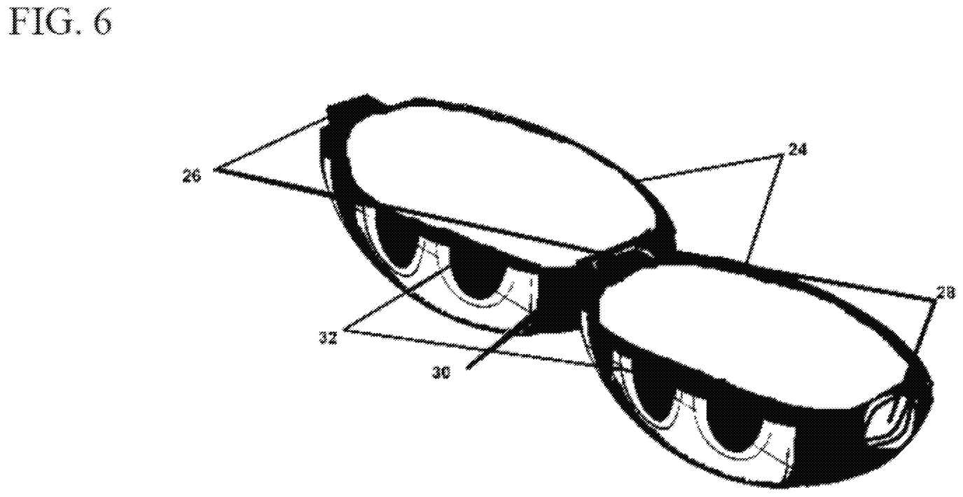

FIG. 5 and FIG. 6 are cross sectional views of the previous described joined set in FIG. 4, represented by 24 and highlighting the grooves within the female locking mechanism 28 as well as the male locking mechanism 26 and the union thereof as 30. Included within the cross-sectional view is the finger grip indentations 32. The joined set pictured has a total of six combined finger grips giving the user a multitude of positions from which to grasp and hold the set in the hand during use. Not pictured is the option of using magnets to secure the lock during use or to replace the male and female locking mechanisms entirely.

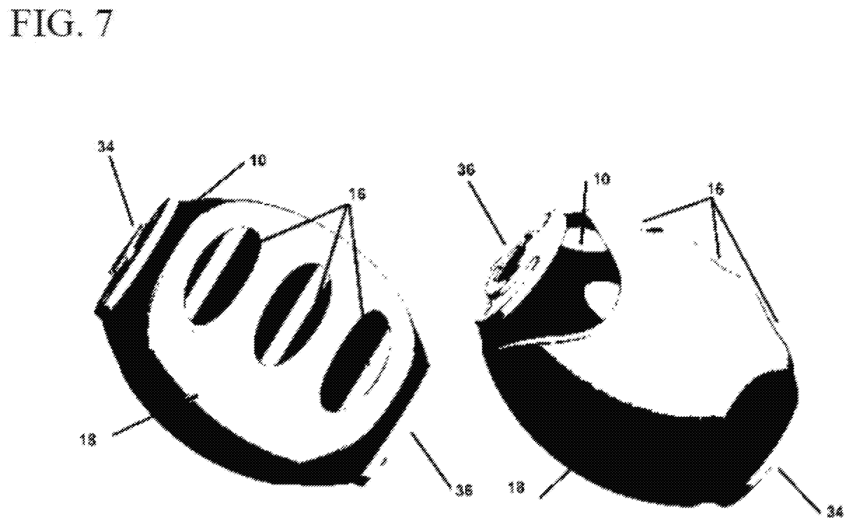

FIG. 7 is an isometric view of an alternative male and female locking mechanism, 34 and 36. The alternative locking mechanism could be more advantageous than the previous suggested solution referred to in FIGS. 1, 2, AND 3. The inventor wishes to identify that many possible interlocking methods exist to join like weight units together and one could argue the benefits and advantages of each; however, the present invention exists not solely on one or two methods of joining weight units together, but rather as a multitude of features and benefits that allow for increasing or decreasing the intensity of held palm centered weight with upper body exercises during aerobic activity. The inventor also envisions a locking mechanism, not pictured here, with a hidden male feature that is flipped out from the weight unit and inserted into a female locking mechanism when joining weight units. Such a system or combination could be more advantageous as it may help to reduce interference from the male locking feature during use with external parts such as headphone wires.

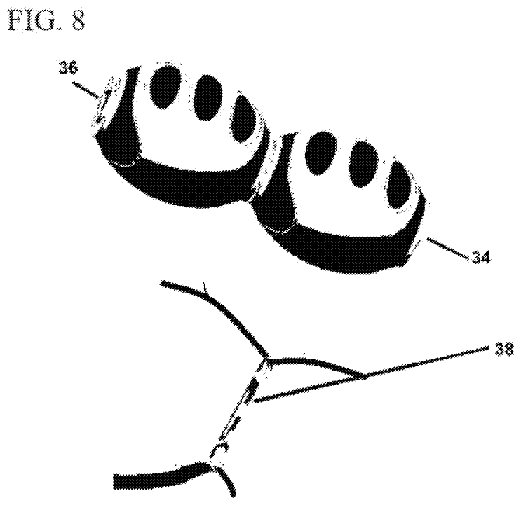

FIG. 8 is both an isometric view and a cross sectional view of the additional locking mechanism example described above and identified as the male locking mechanism 34, the female locking mechanism 36, and the joining of the two as 38.

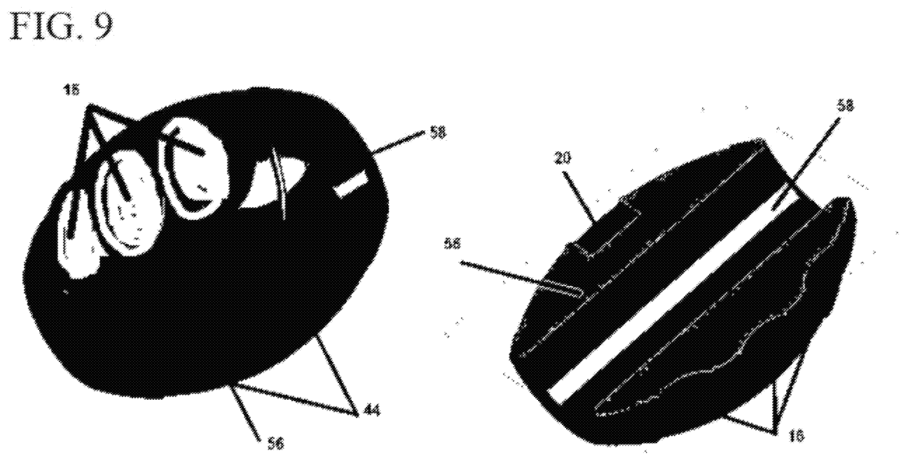

FIG. 9 is a both an isometric view and a cross sectional view of an additional embodiment of the present invention in which weight may be increased or decreased while centered in the palm of the hand during aerobic exercise by use of a master shell weight unit 56 and insert weight segments. Previously described features and elements are continued within the master shell weight unit and insert solution: finger grips 16, and RFID tag 20. The insert weight segment is inserted into the master shell weight unit by means of a center slot or tube 58. The insert weight segment may be locked or anchored into place during use to increase held weight during exercise. The center tube is best illustrated by the cross-section view of the master weight unit 56. Weight inserts are cylinder in nature and therefore easily stored on the body by means of a weight belt, vest, arm band or other method previously discussed. Additionally, this design highlights a wave or raised ridge or line element on the outer portion of the master weight unit to hold the grip sleeve in place during use 44. The weight shell center core may be filled with alternative objects other than a weight insert. For example, a cylinder holding a car key, house key, or a tube of pepper spray may be advantageous for individuals who do not wish to run or walk with a belt or vest holding such items and therefore need a secure location to place these valued objects (not pictured).

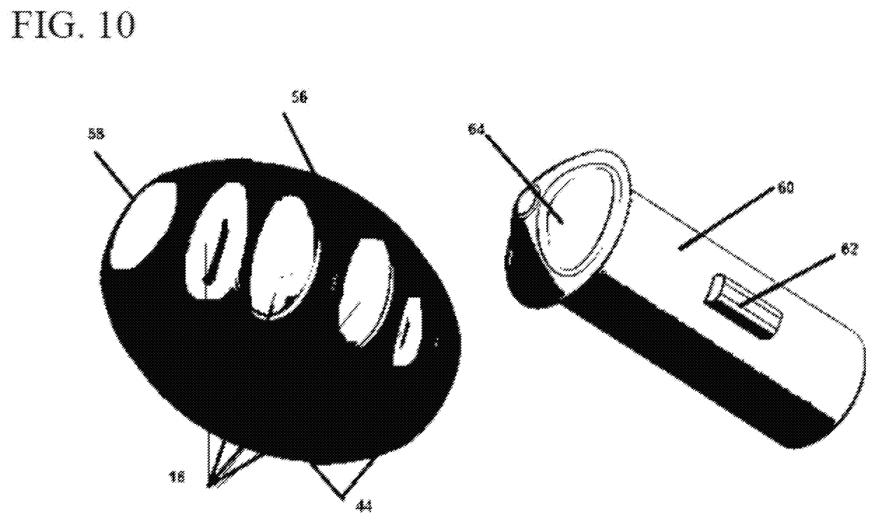

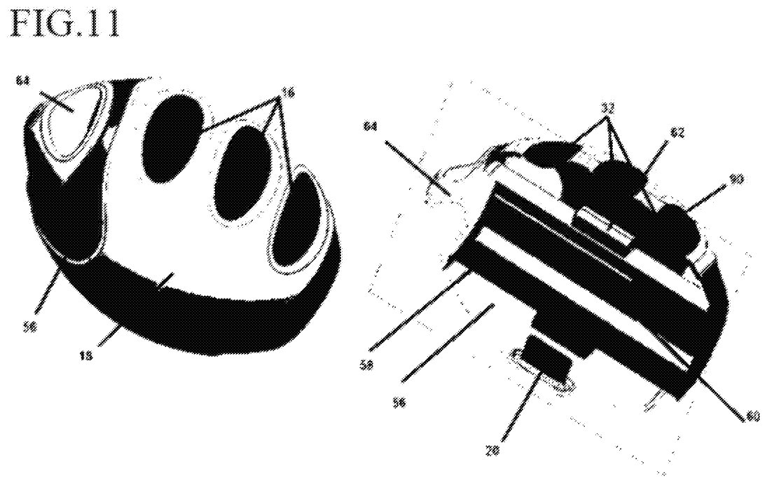

FIG. 10 is an isometric view of both the weight unit shell and the weight unit insert separate, 56 and 60 respectively. In the present view the weight unit insert is identical in length of the insert tube creating a flush end. It is anchored into place by means of twisting or locking the weight insert male feature 62 into a groove or notch located within the master weight shell, not pictured. The weight unit insert has a pointed finger grip head which allows for ease of insertion and providing gripping points from which to twist or spin the weight unit insert into the locking or anchoring position. Additional methods of locking or anchoring the insert weight into the weight shell can be imagined and several examples will be referenced below in FIG. 11.

FIG. 11 is both an isometric view of the weight shell unit and the weight unit insert combined, as well as, a cross section view of the two combined. Depicted in the isometric view are elements previously discussed and referenced as preferred embodiments of the present invention: 16, 18, 20, and 32. Considering the nature of the tube and insert solution, the inventor can imagine many possibilities of locking or anchoring the weight insert segment into the master shell tube such as: a screw and thread system at either ends of the tube and insert, a magnetic core or ring securing the weight within the shell, a magnet or ring on the underneath side of the head of the insert coming in contact with a steel plate or ring, an interlocking edge or hook at any point of the tube or insert that locks or links them together, and a multitude of other methods using locking pins, pressure balls and sockets, latches, push or sliding locking features, or other friction locking mechanisms. The ability to add and remove weight with ease during aerobic exercise is critical to maximize the intensity of the exercises performed during aerobic activity.

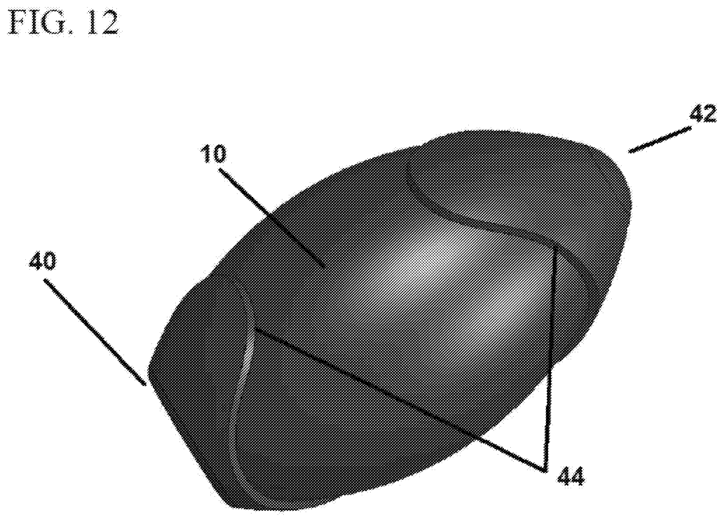

FIG. 12 is an isometric view of a solid weight unit 10, with previously mentioned wave grooves or lines 44, with a flat end little finger rest 40, and a rounded thumb rest at the head of the weight unit 42. Many individuals may not desire the altering weight segments and prefer the traditional singular weight measurement when performing exercises during aerobic activity; however, they would greatly benefit from the use of a palm centered weight unit that does not exceed the circumference of their hand for all of the previously stated reasons when compared to traditional light weight dumbbells, or light weight adjustable dumbbells. The weight unit is specifically designed to be ergonomically held in the center of the palm or hand and takes special attention to the flexibility of not having finger grips cut into the weight's body as this design is to accommodate many finger shapes and sizes as well as a sleeve or glove.

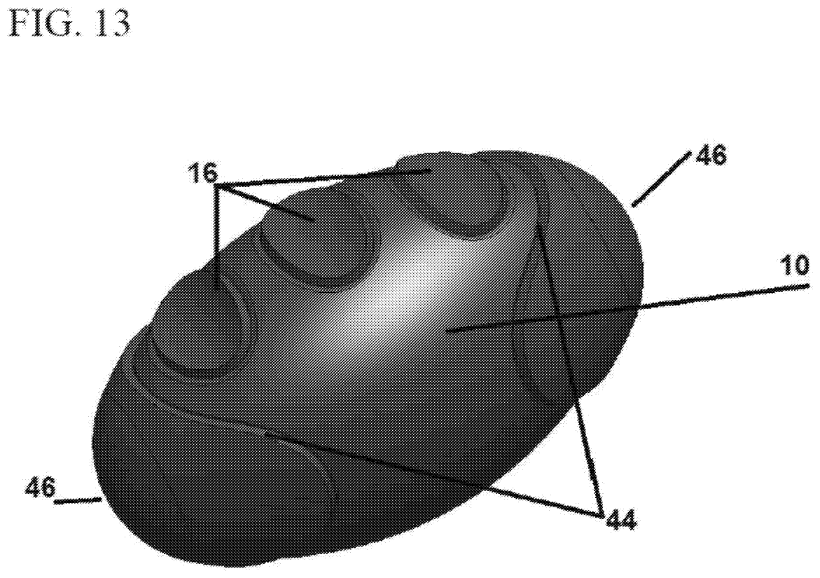

FIG. 13 is an isometric view of a solid weight unit 10, with previous mentioned preferred embodiments 16, and 44 with a new rounded end feature 46. In order to achieve certain desired weights, in a solid unit configuration, special shapes are needed to increase the volume or mass of the palm centered weight unit without exceeding the normal circumference range of the intended user's hand. The largest and heaviest weight units may slightly exceed the intended user's hand circumference and therefore a rounded end may be advantageous to allow for the desired volume with only the smallest amount of weight unit extending past the intended user's hand circumference. During prolonged aerobic exercise, the rounded ends also prevent the aforementioned shortcomings of prior art coming in contact with the body, or other objects such as treadmill side rails, electronic wires, or other items carried on the body such as water bottles or cell phones.

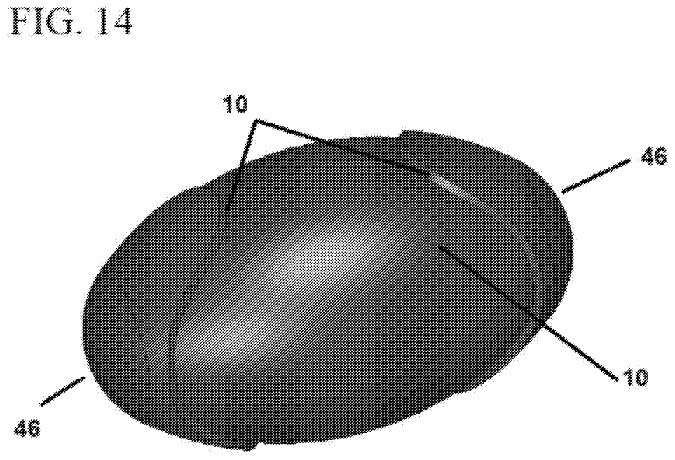

FIG. 14 is a similar isometric view of FIG. 13 without the finger grips 16. The heavier the weight units become, the more varying the hand or finger positioning may become. Therefore, it may be more advantageous to have a rounded end feature 46 without any finger grips, allowing the users to position their fingers at the most functional places to hold the weight comfortably in the center of the hand during prolonged exercise. In addition, users may want to rotate the weight units around in their hands during prolonged use, therefore making a blank finger grip unit most advantageous when compared to those with restrictive pre-molded finger settings.

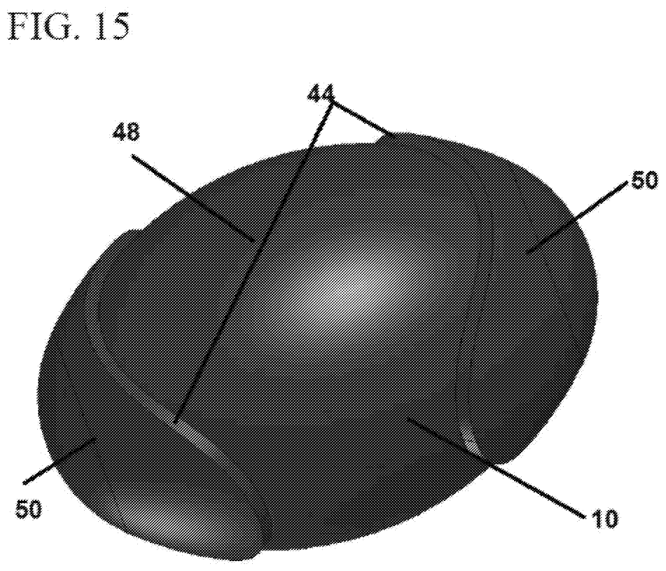

FIG. 15 is a similar isometric view of FIG. 14 with the addition of a large rounded body 48, and large rounded ends 50. Considering most individuals have unique grips and preferences when holding objects, it may also be advantageous for some users to have the option of carrying a weight unit with an increased midsection with larger rounded ends to achieve the desired volume to achieve a heavier weight unit.

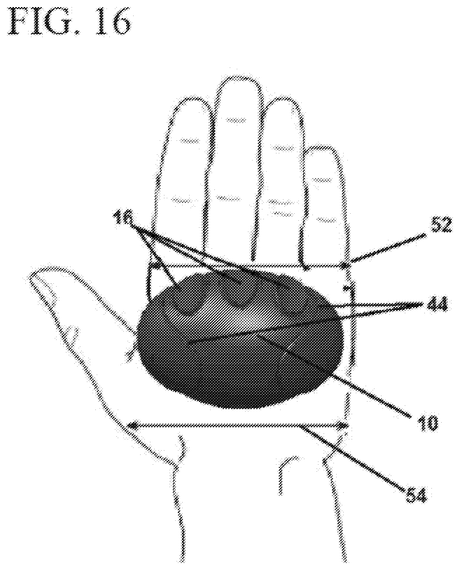

FIG. 16 is a top view of the solid weight unit exhibited in FIG. 13 in an open hand. The weight unit 10, with previously identified preferred embodiments 16 and 44, is depicted as being held across the open hand of an intended user. The open hand circumference at the middle portion of the palm, 52, is smaller than the circumference at the base of the hand which includes the thumb, and outer muscle portion of the hand, 54. Therefore, both dimensions must be considered when developing the correct diameter and circumference of the weight unit. It is most advantageous, for previously mentioned reasons, that the outer most portion of the weight unit must not exceed the total length of the base portion of the palm, 54, but yet lay comfortably across the narrow portion of the palm 52.

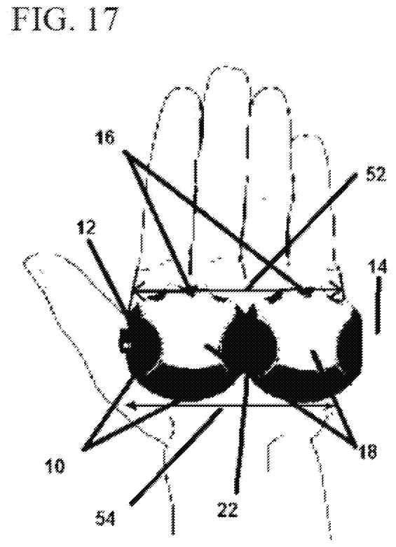

FIG. 17 is a top view of the joined modular weight unit set exhibited in FIG. 3 in an open hand. The modular weight unit set of 10 and 22, with previously identified preferred embodiments 12, 14, 16, and 18, is depicted as being held across the open hand of an intended user. As previously mentioned above, it is most advantageous when joined weight sets do not exceed the circumference of the open hand 52 and fit comfortably in the palm of the hand 54.

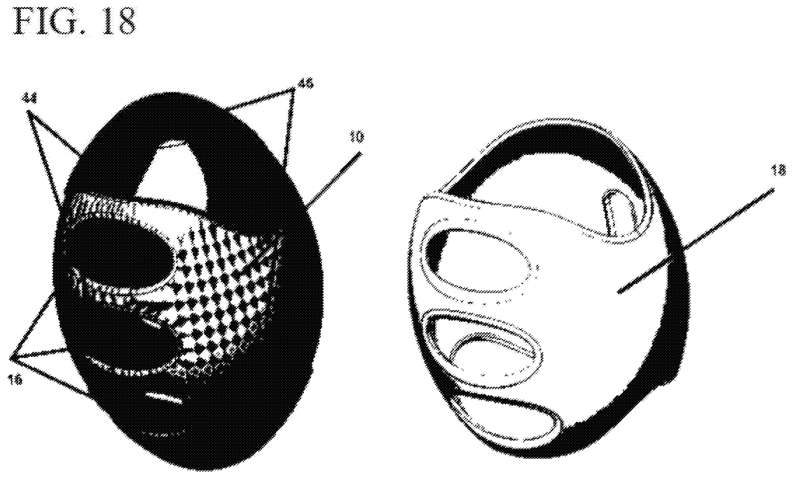

FIG. 18 is an isometric view of the weight unit exhibited in FIG. 13, as 10, and a second isometric view of its corresponding sleeve or glove grip exhibited in FIG. 1, as 18. The single weight unit 10 with its previously identified preferred embodiments 16, 44, and 46, where 44 is the locking or fitting ring for the sleeve or grip glove, 18. The flexible but durable nature of the sleeve or grip glove 18 allows for the ease of application onto the weight unit 10. Users simply pull the sleeve or grip glove around the weight unit and then slide it into place, snug against the wave or line, 44, securing it in place during use. As previously noted, the sleeve or grip glove may be made from varying materials and thicknesses of materials to provide users with a more adjustable fit and holding of the weight unit in the palm of the hand. In addition, the sleeve can be hand washed with soap and water to clean the sleeve or grip glove after use.

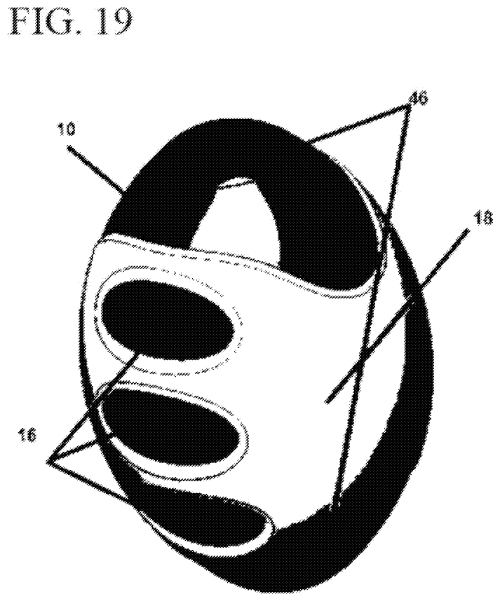

FIG. 19 is an isometric view of the combined two elements identified and described in FIG. 18.

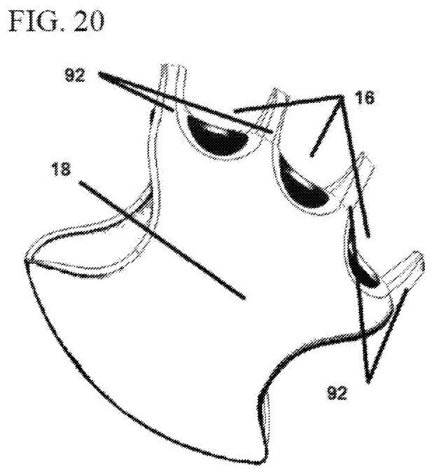

FIG. 20 is an isometric view of the weight sleeve, 18 with extended finger grips, 92 and finger holds 16. The extended finger grips 92 may be more advantageous for some users when performing upper body movements during prolonged aerobic exercise, as the taller grips allow for easier holding of the weight unit in the hand. Any number of taller finger grips may be added to the sleeve to create the desired fit. In addition, the sleeve may also wrap the entire grouping of fingers, enclose any combination of fingers, or enclose any one finger to create a more secure joining of the weight unit and the hand, not pictured.

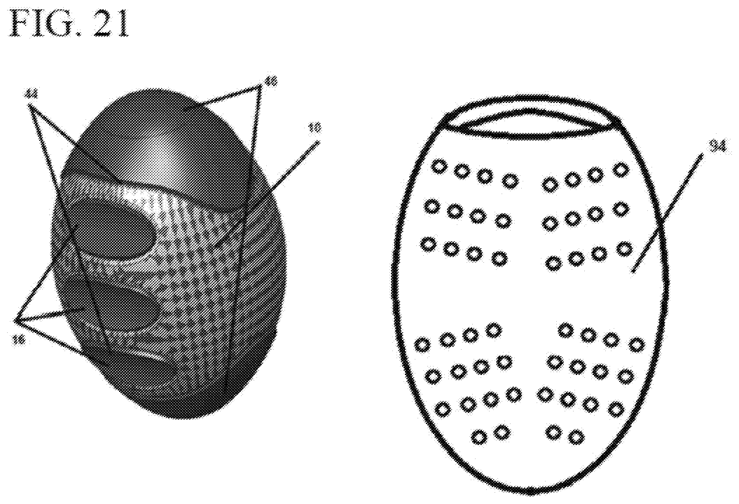

FIG. 21 is an isometric view of the weight unit 10 with previously identified preferred embodiments, 16, 44, 46, and a malleable weight sleeve, 94. The malleable sleeve is intended to be squeezed in the hand and manipulated by the fingers, ostensibly to either help relieve stress and muscle tension or to exercise the muscles of the hand during aerobic activity in combination with held weight unit 10. Material can range from closed-cell polyurethane foam rubber, to a soft aerated or bubbled silicone, to a gel of varying densities inside a rubber or cloth skin. All of these possible materials are intended to provide a resistance to the hand during a squeezing or gripping hand motion. This preferred embodiment is particularly advantageous for those who wish to provide stress or muscle tension relieve in combination with aerobic exercise.

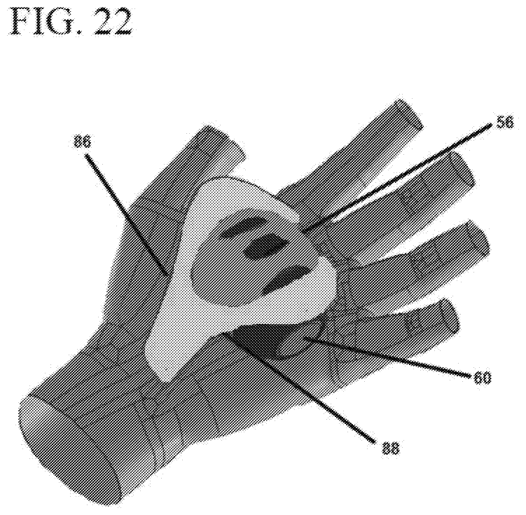

FIG. 22 is an isometric view of a glove complete with weight unit pocket, 86, and 88. As previously identified, fast arm movements may require users to secure the weight units by means of straps, bands, or gloves. This glove example highlights a pocket where the weight unit is secured and housed during exercise, preventing the weight unit from falling or dropping during use. Similar pockets with Velcro strapping may be just as effective as the glove example shown, but considerably more adjustable to hand fit.

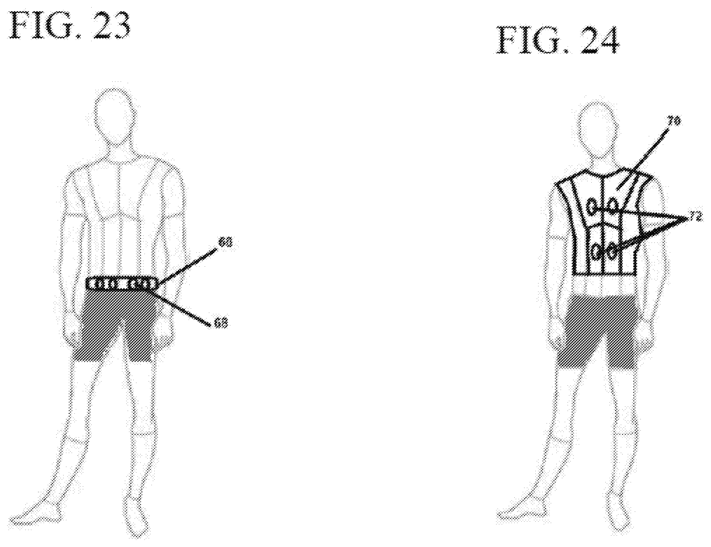

FIG. 23 is an isometric view of a waist belt, 66, where weight units or inserts can be stored and housed in pockets, 68, during aerobic exercise until needed by the user to increase held weight or reduce held weight by storing weight units or inserts on the core of the body. Similar devices may be constructed with additional features such as a water bottle holder, a pocket for keys, a cell phone holder, etc.

FIG. 24 is similar in nature to FIG. 21 depicting a weight storage vest, 70, with storage pockets, 72, for the storing of weight units and inserts or both during aerobic exercise until needed by the user.

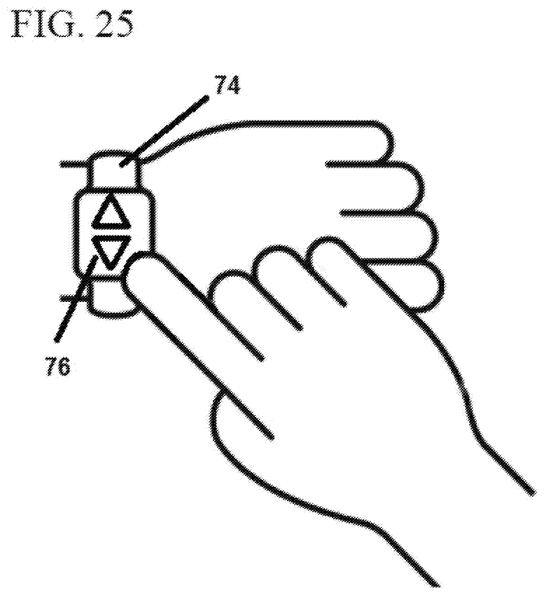

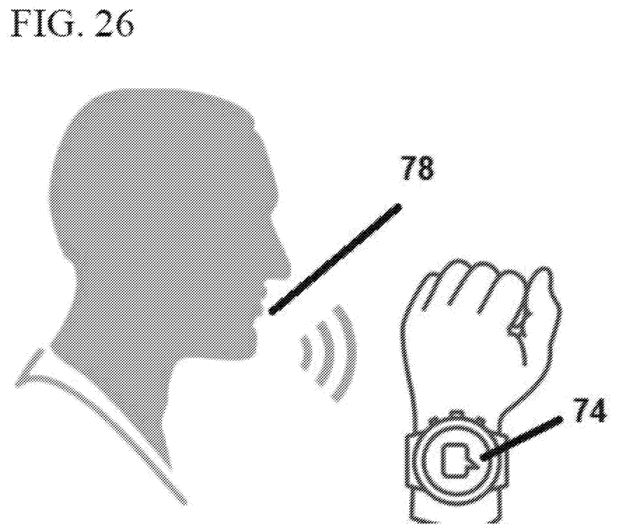

FIG. 25 is an isometric view of a worn wrist tracking device, 74, and an example of a manual touch screen, 76, within to enter the changes in held weight into the tracking device during aerobic exercise. In addition to a touch screen method of inputting held weight values during exercise, other methods may be utilized such as a scrolling ball, push button, or tap bars to identify the starting weight and changes made throughout physical activity. As previously discussed, the ability to simply and easily track held weight values during physical activity is crucial to monitor and track the exertion and intensity of the workout both during and after exercise, as a summary.

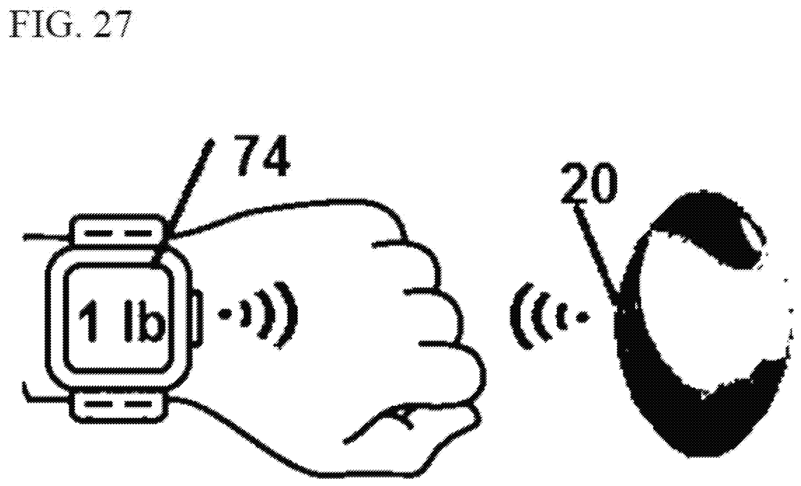

FIG. 26 is an isometric view of a worn wrist tracking device, 74, and an example of changing held weight values by means of voice, 78. It may be more advantageous for some individuals to speak directly into the tracking device to input varying held weight during aerobic exercise, for all the previously noted reasons.

FIG. 27 is a similar view of FIGS. 23 and 24 but highlights the use of the RFID tag to track the varying held weight during physical exercise. Although more expensive to implement, the RFID tag system is a failsafe way to track held weight without the user having to alter their exercise form to manually input the change in weight. The combination of multiple RFID tags to create a new measure of weight would require a tracking or monitoring device capable of reading multiple RFID tags simultaneously.

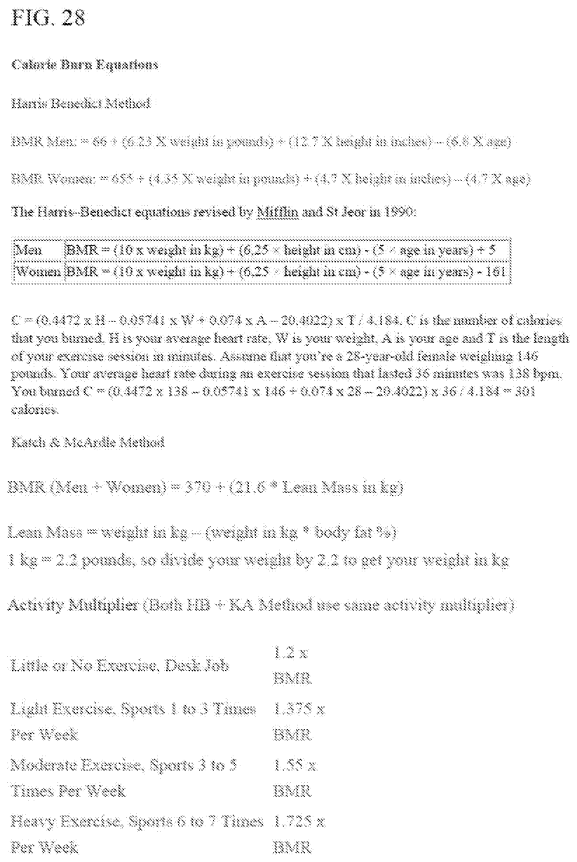

FIG. 28 depicts several established methods of determining calorie burn based on heart rate and other contributing factors. Any of these established methods may be used to track and monitor the user's calorie burn rate during aerobic activity through monitoring devices such as smart watches or heart rate monitors worn on the wrist or forearm as previously mentioned. User's that wish to track the intensity of their workout with the varying held weight against aerobic exercise without will need to create a base line from which the noted methods may be used to evaluate and summarize the differing intensities of workout. These methods of tracking calorie burn and identifying the intensity of exercise may also be used to monitor the user's level of physical fitness, as well as, notify the user when their level of intensity has flat lined or created a new normal level of exertion. This is critical to identify to the user when it is time to increase the held weight, thus creating a higher level of intensity during aerobic exercise.

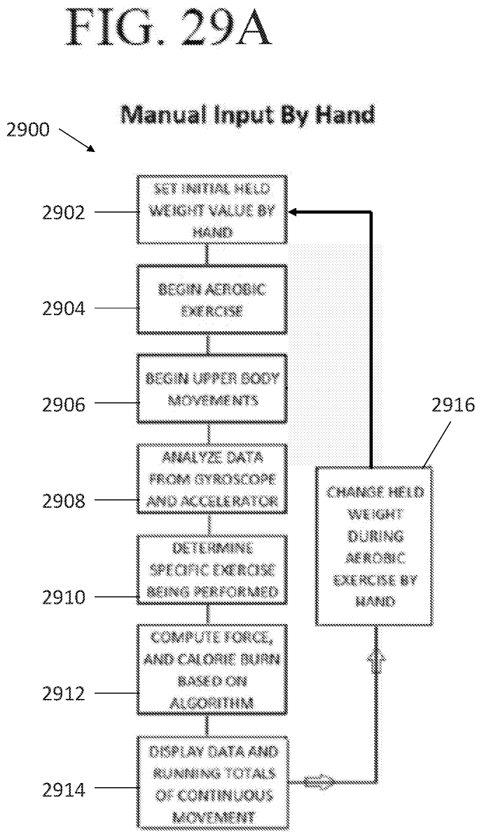

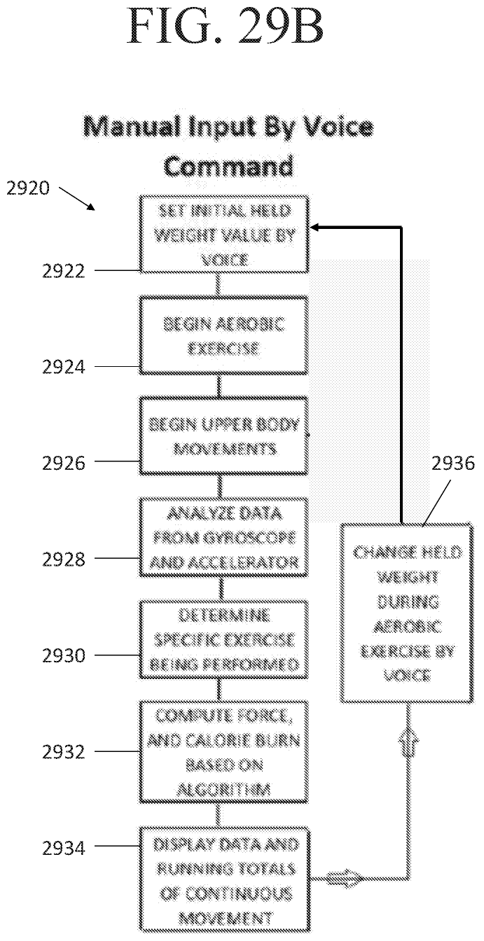

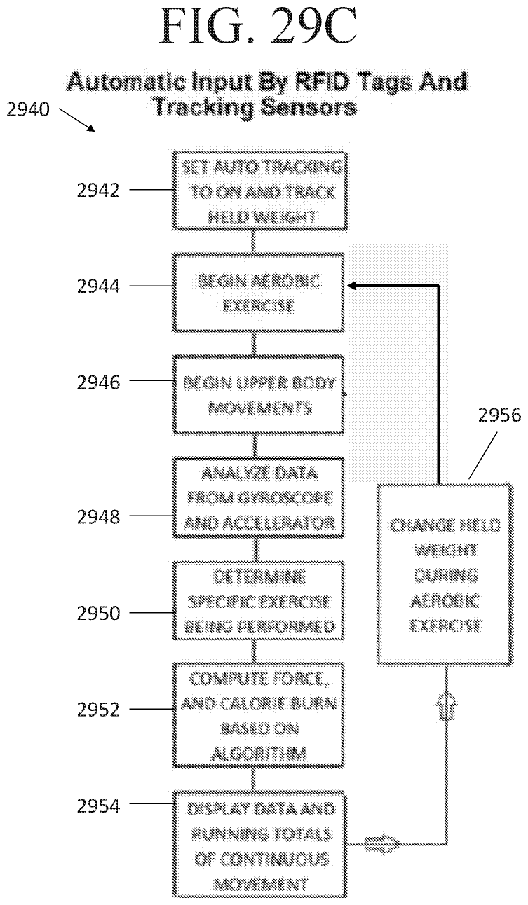

FIGS. 29A-29C are three separate flow chart examples, each depicting how the tracking and monitoring device might calculate and store data both inputted manually or automatically. The flow charts also depict how the tracking or monitoring device might use data from previously noted source FIG. 26 and calculate output and a work out summary of physical activity both continuously and after exercise.

As shown in FIG. 29A, a manual input by hand flowchart 2900 can include various steps. As an initial step 2902, an initial held weight value can be set by hand by either entering a value in a weight value field of an application or by selecting from preset values in a drop-down menu, radio button menu or other menu type. Next, a user can begin an aerobic exercise in step 2904 which can include the application automatically sensing the beginning of movement by the user using sensors such as one or more of gyroscopes, accelerometers or others, or by the user selecting a start exercise button on an application. The user can then begin upper body movements in a subsequent step, which can be similarly sensed or started by selecting an appropriate command in the application in step 2906. The application can then automatically or user-selectively begin analyzing data from the sensor or combination of sensors such as a gyroscope and accelerometer in step 2908. Data analysis can include recording data over a predetermined or ongoing time basis in non-transitory memory and running one or more algorithms using a processor of the analysis device that can be used in step 2910. As such, in step 2910 the application can determine a specific exercise being performed by comparing analyzed data measured by the sensor or sensors from step 2908 with one or more models using at least one algorithm stored in non-transitory memory. The algorithm can be operable to compare the analyzed data with one or more thresholds or exercise profiles in order to determine a best fit or most-likely exercise from a listing of one or more exercises based on one or both of preprogrammed variables or previously measured data samples. Based on the determination of an exercise in step 2910, the application can compute one or more forces, estimate calories burned, perform speed and velocity analysis or perform other analytics using stored algorithms in step 2912. This data can be displayed, via a user interface of the device, in step 2914 and can include periodic or continuous data updates including running totals of measured data. In step 2916, the application can prompt a user to change a held weight during an aerobic exercise or the user can perform this step on their own, which can be confirmed by the user via the user interface. Afterwards, the process can return to step 2902.

As shown in FIG. 29B, a manual input by voice command flowchart 2920 can include various steps. As an initial step 2922, an initial held weight value can be set by voice command by issuing one or more voice commands to an audio receiver, microphone or transceiver of a device that can be run through audio processing software before a weight can be displayed in a weight value field of an application, which can be confirmed by audio confirmation or tactile confirmation using a user interface. Next, a user can begin an aerobic exercise in step 2924 which can include the application automatically sensing the beginning of movement by the user using sensors such as one or more of gyroscopes, accelerometers or others, or by the user selecting a start exercise button on an application. The user can then begin upper body movements in a subsequent step, which can be similarly sensed or started by tactile selecting or voice commanding by announcing an appropriate command to the application in step 2926. The application can automatically or user-selectively begin analyzing data from the sensor or combination of sensors such as a gyroscope and accelerometer in step 2928. Data analysis can include recording data over a predetermined or ongoing time basis in non-transitory memory and running one or more algorithms using a processor of the analysis device that can be used in step 2930. As such, in step 2930 the application can determine a specific exercise being performed by comparing analyzed data measured by the sensor or sensors from step 2928 with one or more models using at least one algorithm stored in non-transitory memory. The algorithm can be operable to compare the analyzed data with one or more thresholds or exercise profiles in order to determine a best fit or most-likely exercise from a listing of one or more exercises based on one or both of preprogrammed variables or previously measured data samples. Based on the determination of an exercise in step 2930, the application can compute one or more forces, estimate calories burned, perform speed and velocity analysis or perform other analytics using stored algorithms in step 2932. This data can be displayed, via a user interface of the device, in step 2934 and can include periodic or continuous data updates including running totals of measured data. In step 2936, the application can prompt a user to change a held weight during an aerobic exercise or the user can perform this step on their own, which can be confirmed by the user via voice command or the user interface. Afterwards, the process can return to step 2922.

As shown in FIG. 29C, an automatic input by RFID tags and tracking sensors flowchart 2940 can include various steps. As an initial step 2942, an initial held weight value can be measured by setting an auto-tracking option of an application to “on” mode by selecting the option in the application using a tactile or voice command. Next, a user can begin an aerobic exercise in step 2944 which can include the application automatically sensing the beginning of movement by the user using sensors such as one or more of gyroscopes, accelerometers or others, or by the user selecting a start exercise button on an application. The user can then begin upper body movements in a subsequent step, which can be similarly sensed or started by selecting an appropriate command in the application in step 2946. The application can then automatically or user-selectively begin analyzing data from the sensor or combination of sensors such as a gyroscope and accelerometer in step 2948. Data analysis can include recording data over a predetermined or ongoing time basis in non-transitory memory and running one or more algorithms using a processor of the analysis device that can be used in step 2950. As such, in step 2950 the application can determine a specific exercise being performed by comparing analyzed data measured by the sensor or sensors from step 2948 with one or more models using at least one algorithm stored in non-transitory memory. The algorithm can be operable to compare the analyzed data with one or more thresholds or exercise profiles in order to determine a best fit or most-likely exercise from a listing of one or more exercises based on one or both of preprogrammed variables or previously measured data samples. Based on the determination of an exercise in step 2950, the application can compute one or more forces, estimate calories burned, perform speed and velocity analysis or perform other analytics using stored algorithms in step 2952. This data can be displayed, via a user interface of the device, in step 2954 and can include periodic or continuous data updates including running totals of measured data. In step 2956, the application can prompt a user to change a held weight during an aerobic exercise or the user can perform this step on their own, which can be confirmed by the user via the user interface. Afterwards, the process can return to step 2944.

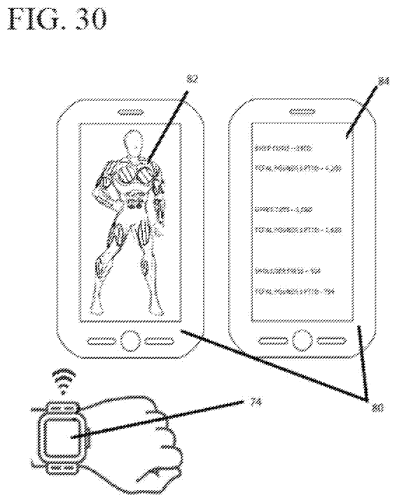

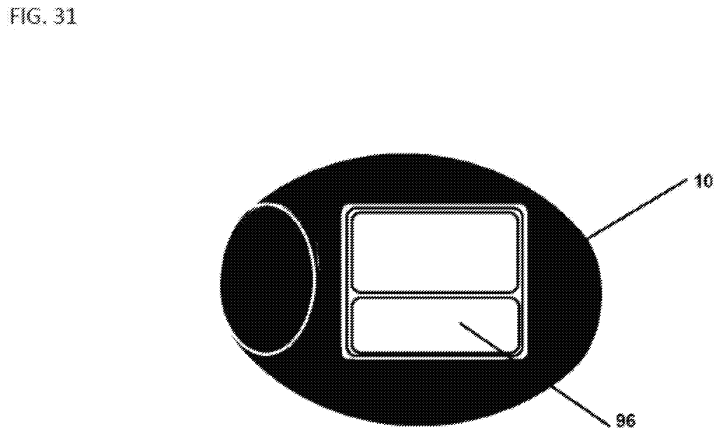

FIG. 30 is an isometric view of a smart phone or tablet, 80, depicting two options of how to review the data and summary information, 82, 84. Information will be transferred from the smart watch, 74, to the tablet by means of a Bluetooth connection or other means of data transfer, wire, cable, etc. Many different formats may be available to choose from to view data summary. The first example is of a total digital, male or female body, 82, displaying the exertion and intensity of the muscle groups used during the period of exercise. The differing in intensity will be identified by a color; for example, red would hold a value of high intensity while green would hold the lowest value of intensity. By touching the muscle group, the user may pull up an alternative view highlighting the specific data generated by that individual muscle group. In addition to a summary page, the digital body can also be used in the training mode where users can identify a specific targeted area and review motions or exercises to target those areas. The second example shows a simple data sheet, 84, to review performance and summary of activity. Additional pages may show full history of exercises with held weight to track performance over a longer period of time to show improvement and strength building.

Activity summary could include but is not limited to:
1. Type of activity performed during aerobic activity—running, walking, hiking, etc.
2. Type of exercises performed during aerobic activity—bicep curls, shoulder press, chest squeeze, upper cuts, triceps extensions, etc. Total amount of upper body exercises performed.
3. Total number of steps or strides achieved during activity.
4. Total number of miles achieved during aerobic activity.
5. Total amount of held weight by each exercise for the duration of the aerobic activity.
6. Total amount of held weight by duration of aerobic activity.
7. Total amount of held weight this week, month, and year.
8. Total number of miles performed this week, month, and year.
9. Total calories burned during activity.
10. Total calories increased with the use of the weight units and exercises versus just aerobic activity alone.

FIG. 31 is an isometric view of the hand held weight unit 10. The weight unit 10 has a center recess or indentation area allowing for the insertion of a sensor package 96. The centrally located sensor package 96 can be equipped with various real time tracking devices such as gyroscopes or multi-directional accelerators. The purpose of the centrally located sensor is to collect data from a fixed home location of varying points across the X, Y, and Z axis to locate and track the weight unit during exercises that are prescribed by a physical therapist for rehabilitation purposes. Tracking and real time monitoring of the location of the held weight unit allows users to perform accurate movements against a predetermined exercise routine.

There are many existing low power three-axis angular rate sensors or digital gyroscope devices on the market today that feature excellent scale accuracy from ±100 to ±2000 dps to track motion. Some of the manufactures and suitable products are: STMicroelectronics models L3GD20HTR and the L2G2ISTR, the Maxim Integrated model Max 21000, NXP Semiconductors model FXAS21002C, and the InvenSense model ITG-3200. In addition to the various digital gyroscopes that detect and track orientation of the weight unit by using the principles of angular momentum, an accelerometer is used to detect and track liner acceleration, tilt, shock, and vibration through force exerted on the weight unit by gravity. Some of the manufacturers and suitable products are: Analog Devices models ADXL700, ADXL363, ADXL375, and ADXL377, and the NXP Semiconductors model MMA8452Q. These two sensors, gyroscopes and accelerators, combine to obtain the motion of the weight unit in a 3-d space to a high degree of accuracy. The data generated by these sensors is sent to a smart phone, tablet, or other digital display device by means of short range wireless connectivity. An example of this technology would be the Dual Mode Bluetooth technology, or the Bluetooth BR/EDR technology. A rechargeable lithium ion battery supplies power to the removable sensor package, 96. The sensor package 96 houses all the electronic components to transfer data from the sensors to the digital display unit. The sensor package's shape may have a straight, curved or angled face to better fit into the various weight units without disrupting the natural grip of intended users, not pictured.

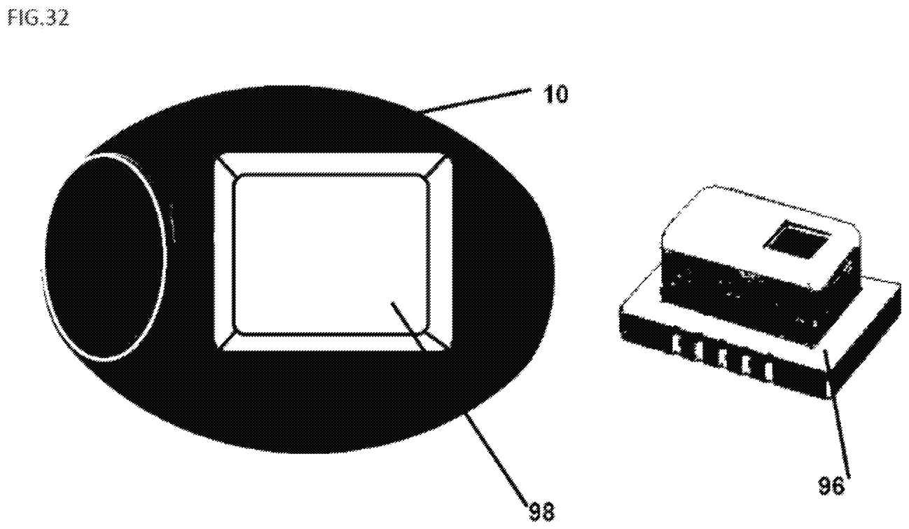

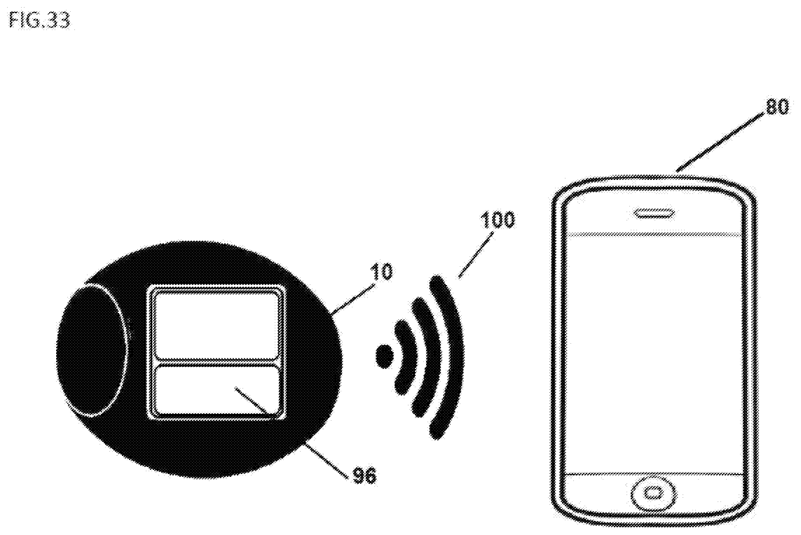

FIG. 32 is an isometric view of the hand held weight unit 10 and the sensor package 96 separately. The center insert area 98 is identical across multiple sized weight units 10, not pictured. By standardizing the sensor unit 96 and the weight recess area 98 across a multitude of weight shapes and weight increments, it allows for the removal and re-insertion of the sensor package 96 across many prescribed therapeutic weights of varying intensities over weeks and even months of prescribed therapeutic exercises. For example, as an individual performs his or her physical therapy over time, the body will regain its strength and motion requiring the addition of heavier weight units with an even more difficult set of prescribed motions to reach the ultimate goal of recovery.

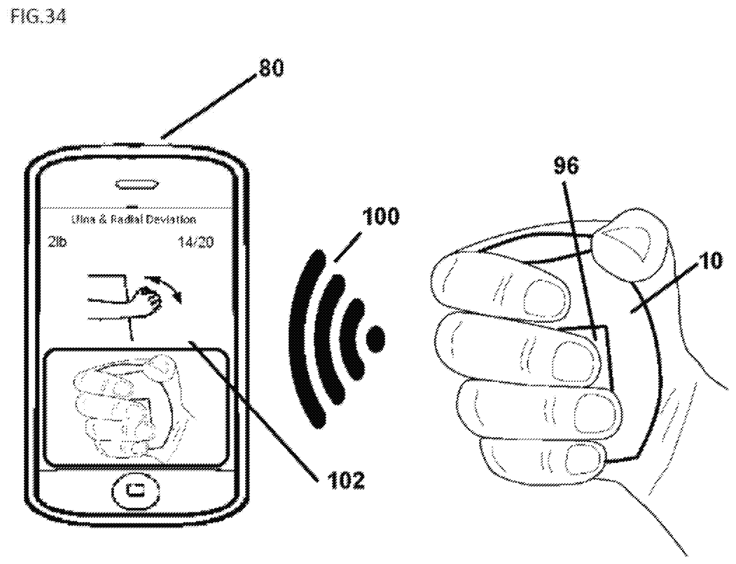

FIG. 33 is an isometric view of the weight unit 10 with inserted sensor unit 96 combined into a set with the wireless connective 100 between the sensor unit 96 and the smart phone, tablet, or another display unit 80. The sensor unit is equipped with a rechargeable power source, wireless or Bluetooth connection, microprocessor or chip, and sensors that may include any combination of a gyroscope and or multi-directional accelerators. The smart phone, tablet or another digital display device receives, real time data points from the sensor unit and translates the data into a digital representation of the movement made by the user. The digital display will show the user the accuracy of the performed motion and suggest corrective action if needed as well as track number of movements against a prescribed therapeutic exercise routine. The digital display unit will also act as a virtual therapist gathering data, asking predetermined questions based on the performance, accuracy, and perceived level of difficulty, as well as set goals for the user based upon results. At any time during the physical therapy, the user may stop the exercise routine and identify a pain or discomfort level from 1-10 directly at the location of the injury on the digital display and immediately upload the information directly to the licensed therapist for further instruction. It is critical that in home therapy by the user must be monitored and accurate to not further damage the injury during unsupervised exercise.

FIG. 34 is a similar isometric view of FIG. 33 depicting an example of the digital display with the corresponding held weight referenced hand motion. In this example, the weight unit is identified as a two-pound weight, the prescribed exercise or range of required motion is identified by a series of directional arrows along with a digital hand which performs the correct motion required, and lastly a number of expected 20 reputations against actual number of 14 performed. Many other additional data points may be shown on the digital display through selecting a customized profile by the user or therapist. Examples of custom data points can include, but are not limited to: time to perform each therapeutic exercise, accuracy of movement, rest period in-between each activity, total weight lifted, total exercises performed, next scheduled therapy session, and remaining days of therapy.

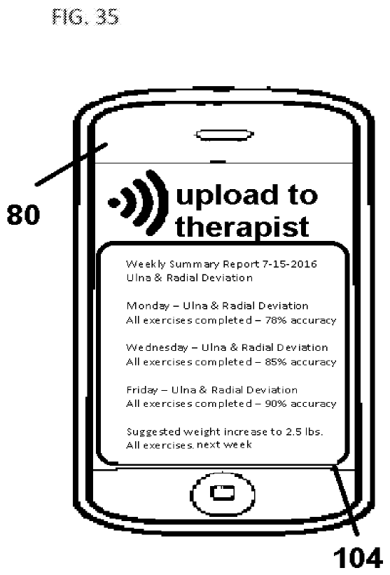

FIG. 35 is an isometric view of the smart phone example 80 with a detailed summary 104 of the completed therapy exercises for the week of Jul. 16, 2016, and an “upload” to therapist button that resembles the wireless communication signal. This example of the summary page 104 includes: name of injury and therapy performed, compliance by day, and accuracy of movement each day. This summary page allows both the user and the therapist to communicate over a simple format to monitor and track performance. In this example, the user is showing a steady increase in accuracy of movements as well as a 100% completion rate during the week. This example indicates to the therapist that the user is grasping the technology, performing all movements well, and may be in need of a weight increase to move to the next level of therapy. If by chance the weekly report log 104 indicated only a 50% accuracy level with only 60% of required movements completed for the week, the therapist may increase the office visits and retrain the user on the technology and importance of compliance with the at home portion of therapy. The therapist may also look further into the weekly reports and watch actual performed movements to determine where the problem exists and identify an action plan to fix the problem.

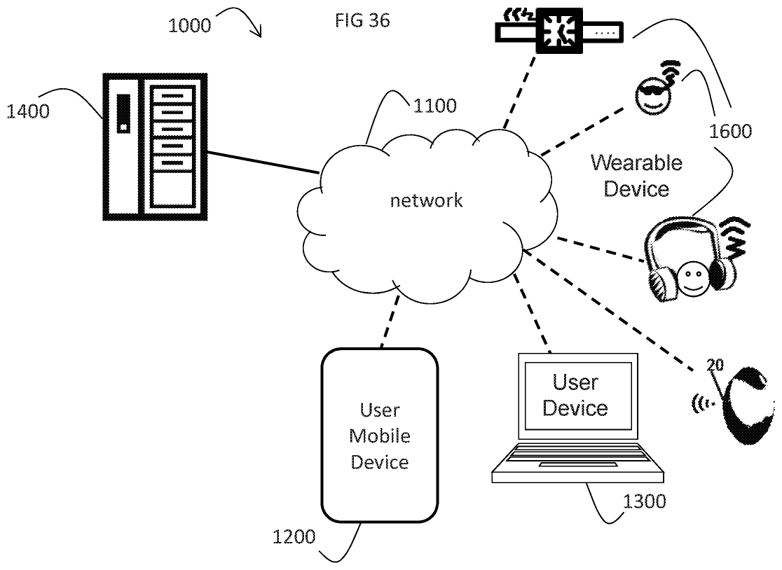

FIG. 36 is an example embodiment of a network system architecture diagram 360. As shown in the example embodiment, a server system architecture 360 can include a combination wired and wireless network communication network 362 and can be private, public (e.g. the Internet and/or a cellular-based wireless network, or other network) or a combination thereof. At least one server 364 can be communicatively coupled with network 362 and can include applications and operating systems distributed on one or more physical servers. Server 364 can include one or more processors, memory banks, operating systems, input/output interfaces, power supplies and network interfaces, as are known in the art or later developed. A plurality of wearable end user devices 366 can be communicatively coupled to network 362. Examples of wearable user devices include, smartwatches, smart-bracelets, smart-glasses, smart-headphones and others and can each have user interfaces, processors, memory, power, communication transceivers and other components. User mobile devices 368 can be communicatively coupled to network 362 and can include smartphones, tablets, phablets, PDA's and others. User devices 370 can be communicatively coupled to network 362 and can include desktop or laptop computer devices and other devices with computing capability, memory, processors and other components and hardware and software, network interfaces and so on. Hand held modular weight units 10 (also referred to herein as a monitoring exercise device) can be communicatively coupled to network 362 and include power and network connectivity, sensors and other components as described herein.

In various embodiments, wearable devices 366, user mobile devices 368, user devices 370 and hand held modular weight units 10 can variously communicate with each other and server 364 bi-directionally and uni-directionally via network 362 and can used by various users, such as athletes, patients, coaches, technicians, administrators and other users as appropriate. As such, system architecture 360 provides users the ability to access various features and functions of the overall system, where servers 364 and the various other devices can be operable to interface with websites, webpages, web applications, web portals, social media platforms, advertising platforms and others, as appropriate.

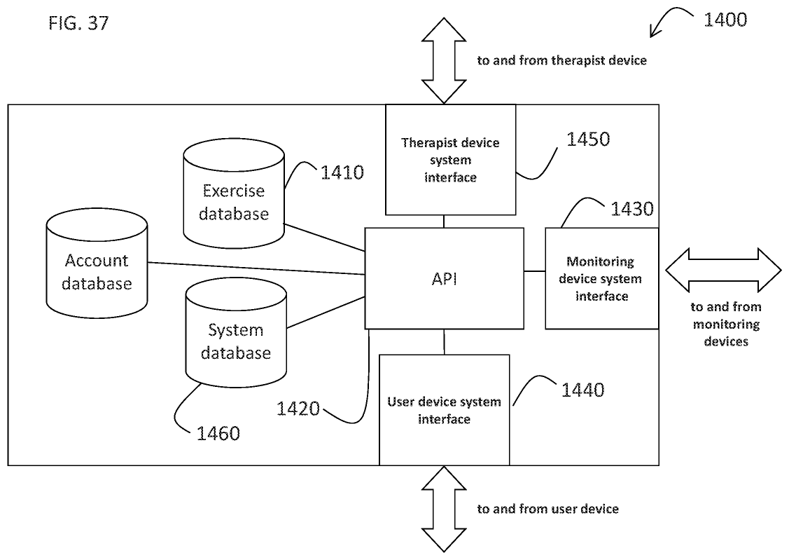

FIG. 37 is an example embodiment of network connected system server 364, in the form of a system architecture diagram. As shown in the example embodiment, a server 364 can include at least one user device interface 380 implemented with technology known in the art for communication with user devices (e.g. user mobile device 368, user device 370 and wearable devices 366 of FIG. 36) as described elsewhere herein, known in the art or developed in the future. Server 364 can also include at least one web application server system interface 382 for communication with web applications, websites, webpages, web portals, social media platforms advertising platforms and others. Additionally, server 364 can include at least one monitoring exercise device interface 384 implemented with technology known in the art for communication with monitoring exercise devices (e.g. monitoring exercise device 10 of FIG. 36) as described elsewhere herein, known in the art or developed in the future.

Server 364 can also include an exercise database 388, which can be a database as known in the art or later developed, stored in non-transitory computer readable memory. Exercise database 388 can include algorithms, data and other information related to particular exercises and used to identify such exercises. Server 364 can also include an account database 390, which can be a database as known in the art or later developed, stored in non-transitory computer readable memory. Account database 390 can include algorithms, data and other information related to particular user accounts for system users. Examples of account information can be usernames, passwords, statistics, identifying information, data logs, preferences and others. Additionally, Server 364 can also include a system database 392, which can be a database as known in the art or later developed, stored in non-transitory computer readable memory. System database 392 can include algorithms, data and other information related to system functionality, such as descriptions, blog postings, recommendations, webpage layouts and other information. The databases can be implemented with technology known in the art such as relational databases and/or object oriented databases or others

Each of interfaces 380, 382 and 384 and databases 388, 390 and 392 can be coupled with an application program interface (API) 386 such that can functionally communicate, as is typically understood in the art. API 386 can instruct the databases to store (and retrieve from the databases) information such as user account information, exercise information, associated account information, instructional information, warranty information, communication information or others as appropriate.

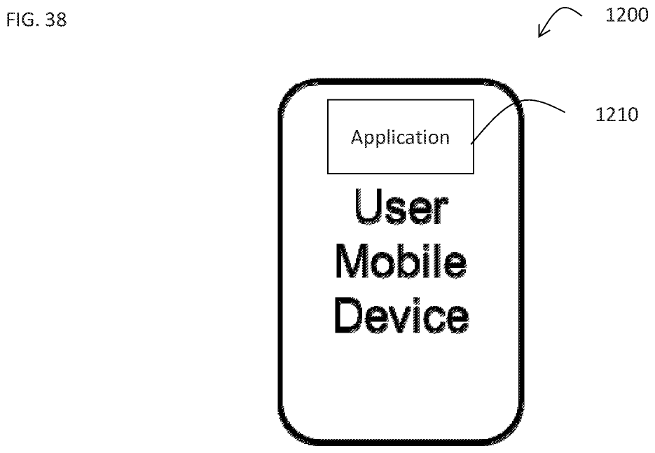

FIG. 38 is an example embodiment of a user mobile device 368 architecture diagram. In an example embodiment, user mobile device 368 can include a network connected application 394 that is installed in, pushed to or downloaded to the user mobile device and stored in non-transitory computer readable memory. In many embodiments, user mobile devices are touch screen devices that can display graphical user interfaces. Applications can include exercise, account and communication information for viewing and downloading on a user device, communicating with other users, saving information locally or remotely, controlling monitored exercise equipment remotely or locally and others.

Mobile applications, mobile devices such as smart phones/tablets, application programming interfaces (APIs), databases, social media platforms including social media profiles or other sharing capabilities, operating systems, load balancers, web applications, page views, networking devices such as routers, terminals, gateways, network bridges, switches, hubs, repeaters, protocol converters, bridge routers, proxy servers, firewalls, network address translators, multiplexers, network interface controllers, wireless interface controllers, modems, ISDN terminal adapters, line drivers, wireless access points, cables, servers, power components and others equipment and devices as appropriate to implement the methods, systems and devices are also contemplated.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. An ergonomic weight unit for aerobic exercise that is centered in the hand without exposure beyond the circumference of the intended user's hand comprising:
a solid weight unit of constant weight;
a modular weight unit of constant weight with interlocking features to either join or separate units for increasing or decreasing held weight; and
a master shell weight unit of constant weight with interlocking insert blocks or tubes to either join or separate units for increasing or decreasing held weight.

2. The ergonomic weight unit of claim 1 further comprises finger grips or indentations on the weight unit to allow for gentle holding thereof while centering the weight unit in a palm of the hand.

3. The ergonomic weight unit as of claim 1 further comprises a rubber, silicone, or similar durable but flexible material molded into the weight unit's shape and used as a glove or sleeve to wrap the weight unit in its entirety or partially to provide a better grip as well as an anti-slip device especially from sweat build up during aerobic activity.

4. The ergonomic weight unit of claim 1, wherein units and inserts are stored on a body of the weight unit during aerobic activity using a waist belt, an arm band, a vest, or an ankle band to hold the weight unit on the body of the user.

5. The ergonomic weight unit of claim 1 further comprises a RFID tag imbedded into the weight unit for automatic tracking and monitoring of held weight during aerobic exercise.

6. The ergonomic weight unit of claim 1 further comprises a wrist or forearm worn monitoring device for tracking varying held weight during aerobic exercise using manual input or voice input from the user.

7. The ergonomic weight unit of claim 1 wherein multiple casted materials are used to achieve a desired weight including a lead based center of the weight unit surrounded by casted steel or other metals such as cast iron.

8. The ergonomic weight unit of claim 1 further comprises a groove or line wherein a glove or sleeve attaches for a tight fit, therefore holding the glove or sleeve in place during aerobic activity.

9. An ergonomic weight unit for aerobic exercise that is centered in the hand without exposure beyond the circumference of the intended user's hand comprising:
a modular weight unit of constant weight with interlocking features to either join or separate units for increasing or decreasing held weight;
a master shell weight unit of constant weight with interlocking insert blocks or tubes to either join or separate units for increasing or decreasing held weight; and
magnets for securing joining modular weight units or master shell and insert tube together to form one unit of weight and volume for use during aerobic exercise.

10. A monitoring or tracking device comprising:
a touch screen visual display with a digital, male or female body for displaying a workout summary by exercise and by muscle groups, total intensity of workout by use of color identification marks on the digital body identifying low, medium, and high levels of intensity by color.

11. The monitoring or tracking device of claim 10, wherein the monitoring or tracking device is a smart device, a smart phone, or a tablet.

12. The monitoring or tracking device of claim 10 further teaches proper technique, form, and exercises by muscle group in a teaching mode to instruct users proper way to perform upper body exercises during aerobic activity.

13. The monitoring or tracking device of claim 10 further displays actual movements of the user against movement patterns of proper form and technique of the exercise.
